SEQUENCE LISTING (15 sequences)

SEQ ID NO: 1 - DNA, 24 bp, Photinus pyralis
ttatgtttttggcgtcttccatgg

SEQ ID NO: 2 - DNA, 26 bp, Photinus pyralis
gccaaaaacataaaccatggaagact

SEQ ID NO: 3 - DNA, 25 bp, Photinus pyralis
cctcttacctcagttacaatttata

SEQ ID NO: 4 - DNA, 21 bp, Photinus pyralis
cuuacgcugaguacuucgatt

SEQ ID NO: 5 - DNA, 21 bp, Photinus pyralis
ucgaaguacucagcguaagtt

SEQ ID NO: 6 - DNA, 21 bp, Escherichia coli
gcgauaagucgugucuuactt

SEQ ID NO: 7 - DNA, 21 bp, Escherichia coli
guaagacacgacuuaucgctt

SEQ ID NO: 8 - DNA, 21 bp, Homo sapiens
agggcaacuuccagaccautt

SEQ ID NO: 9 - DNA, 21 bp, Homo sapiens
auggucuggaaguugcccutt

SEQ ID NO: 10 - DNA, 21 bp, Mus musculus
cacucagucucuaagggcutt

SEQ ID NO: 11 - DNA, 21 bp, Mus musculus
agcccuuagagacugagugtt

SEQ ID NO: 12 - DNA, 21 bp, Aequorea victoria
gaacggcaucaaggugaactt

SEQ ID NO: 13 - DNA, 21 bp, Aequorea victoria
guucaccuugaucccguuctt

SEQ ID NO: 14 - DNA, 21 bp, Variola virus
cgggauaucuccagacggatt

SEQ ID NO: 15 - DNA, 21 bp, Variola virus
uccgucuggagauaucccgtt

(19) United States
(12) Patent Application Publication
(10) Pub. No.: US 2003/0143204 A1
(43) Pub. Date: Jul. 31, 2003

(54) Inhibition of RNA function by delivery of inhibitors to animal cells

(76) Inventors: David L. Lewis, Madison, WI (US); James E. Hagstrom, Madison, WI (US); Hans Herweijer, Madison, WI (US); Aaron G. Loomis, Prairie du Sac, WI (US); Sean D. Monahan, Madison, WI (US); Vladimir S. Trubetskoy, Madison, WI (US); Jon A. Wolff, Madison, WI (US)

Correspondence Address: Mark K. Johnson, Mirus, 505 South Rosa Road, Madison, WI, 53719, US

(21) Appl. No.: 10/186,757

(22) Filed: Jul. 1, 2002

Related U.S. Application Data
(63) Continuation-in-part of application No. 09/917,154, filed on Jul. 27, 2001.
(60) Provisional application No. 60/315,394, filed on Aug. 27, 2001.
(60) Provisional application No. 60/324,155, filed on Sep. 20, 2001.

(51) Int. Cl. A61K 48/00; C12N 15/86

(52) U.S. Cl. 424/93.2; 435/456

(57) ABSTRACT

Described is a process for delivering an inhibitor directed against an expressible nucleic acid sequence in a mammal to inhibit RNA function. An RNA function inhibiting sequence that is specific to the expressible nucleic acid sequence in the mammal is made and inserted into a blood vessel in the mammal. The inhibitor is delivered to a cell wherein expression of the nucleic acid sequence is inhibited.

This Patent Application is related to pending U.S. provisional patent applications 60/315,394 filed Aug. 27, 2001 and 60/324,155filed Sep. 20, 2001.

### FIELD

The present invention generally relates to inhibiting gene expression using double-stranded nucleic acids. Specifically, gene expression is inhibited relating to bacterial infection such as anthrax or related to a viral infection such as small pox.

### BACKGROUND

Infectious agents represent a serious threat to human health and national security because they may be used in bioterrorist attack or biological warfare. These agents include known viruses such small pox and bacteria such as anthrax as well as new unknown agents. Several aspects of biological warfare underscore the need to develop new highly adaptable, easily synthesized countermeasures. For example, while small pox, a variola virus, has been eradicated from the population worldwide, it has been developed as a biological warfare agent in the former Soviet Union and likely other nations and terrorist organizations¹. The Soviets had a large secret program to develop viral biological warfare agents at the All-Union Scientific Research Institute of Molecular Biology in Koltsovo, Siberia (known as “Vector”)¹. Objectives of the project included increasing virulence and pathogenicity of viruses and the production of massive mounts of the virus. Particularly virulent strains of variola were sequenced in order to identify genes responsible for the virulence with the goal to combine these genes into a single super virus. Another project was to develop a variola virus resistant to commonly-used vaccines. The Soviet Vector program furthermore developed new genetically engineered virus hybrids including combining the ebola virus with variola virus¹. The ability to engineer more deadly viruses was exemplified by a recent Nature article reporting that a hybrid HIV/SIV became resistant in immunized monkeys via a single mutation².

It is unlikely that current vaccines and drugs would be effective in treating an outbreak of these new pathogens. Furthermore, traditional approaches to developing antimicrobial vaccines or small molecule drugs are too slow to be effective in providing countermeasures to the purposeful release of such infectious agents. Therefore, new technologies are needed in order to combat this emerging threat. One such technology is the in vivo delivery of siRNA to inhibit gene expression of the pathogens. SiRNA technology has an advantage over traditional drugs in that it is more readily adapted to new mutated or engineered infectious agents. A new infectious agent can be quickly sequenced and siRNA molecules synthesized to combat new biological weapon threats. Delivery of the siRNA would remain the same regardless of the specific sequence. Pathogen genes for transcription, replication, or virulence may be targeted to decrease the contagiousness and boost survival of infected individuals. SiRNA treatment of poxviral or other infection furthermore has the potential to delay onset of major disease until immunoprotection has been acquired.

Traditional vaccine approaches to combat certain infections have also proven inadequate to slow or prevent a number of natural diseases, including malaria, AIDS, herpes, dengue fever and some forms of viral hepatitis. These diseases are also candidates for siRNA therapy.

RNA interference (RNAi) describes the phenomenon whereby the presence of double-stranded RNA (dsRNA) of sequence that is identical or highly similar to a target gene results in the degradation of messenger RNA (mRNA) transcribed from that target gene³. However, researchers have been pessimistic about applying RNAi to mammalian cells because exposing mammalian cells to dsRNA of any sequence triggers a global shut down of protein synthesis.

The introduction of long dsRNA into mammalian cells is known to induce an interferon response which leads to a general block in protein synthesis and leads to cell both by both non-apoptotic and apoptotic pathways⁴. In fact, studies performed using mammalian cells in culture indicate that introduction of long, dsRNA does not lead to specific inhibition of expression of the target gene^{5,6}. A major component of the interferon response to dsRNA is the dsRNA-dependent protein kinase PKR, which phosphorylates and inactivates elongation factor elF2a. In addition, dsRNA induces the synthesis of 2′-5′ polyadenylic acid leading to the activation of the non-sequence specific RNase, RNaseL⁷. While, it has previously been demonstrated that long dsRNA can be used to inhibit target gene expression in mouse oocytes and embryos^{8,9}, it is likely that the interferon response pathway is not present at this early developmental stage in these cells.

More recently, it has been found that RNAi is likely mediated by short interfering RNAs (siRNAs) of approximately 21-25 nucleotides in length which are generated from the input dsRNAs^{10,11,12,13,14}. It has also been found the short dsRNA does not appear to induce the interferon response in mammalian cells in culture. One reason for this may be that these siRNAs are too small to activate PKR^{15,16}. Finally, it has been shown that siRNA <30 bp do induce RNAi in mammalian cells in culture^{17,18}.

The ability to specifically inhibit expression of a target gene by RNAi has obvious benefits. For example, RNAi could be used to generate animals that mimic true genetic “knockout” animals to study gene function. In addition, many diseases arise from the abnormal expression of a particular gene or group of genes. RNAi could be used to inhibit the expression of the genes and therefore alleviate symptoms of or cure the disease. For example, genes contributing to a cancerous state could be inhibited. In addition, viral genes could be inhibited, as well as mutant genes causing dominant genetic diseases such as myotonic dystrophy. Inhibiting such genes as cyclooxygenase or cytokines could also treat inflammatory diseases such as arthritis. Nervous system disorders could also be treated. Examples of targeted organs would include the liver, pancreas, spleen, skin, brain, prostrate, heart etc. The ability to safely delivery siRNA to mammalian cells in vivo has profound potential for the treatment of infections and diseases as well as drug discovery and target validation.

Several aspects of current pharmaceutical research and therapeutic treatment are candidates for siRNA technology. For the purposes of target validation, gene inactivation allows the investigator to assess the potential therapeutic effect of inhibiting a specific gene product.

Expression arrays can be used to determine the responsive effect of inhibition on the expression of genes other than the targeted gene or pathway. Other methods of gene inactivation, generation of mutant cell lines or knockout mice suffer from serious deficiencies including embryonic lethality, expense, and inflexibility. Also, these methods frequently do not adequately model larger animals. Development of a more robust and easily applicable gene inactivation technology that can be utilized in both in vitro and in vivo models would greatly expedite the drug discovery process.

### BRIEF DESCRIPTION OF FIGURES

FIG. 1. siRNA is efficiently delivered to multiple tissue types in mice in vivo and the delivered siRNA is highly effective for inhibiting target gene expression in all organs tested.

FIG. 2. Intravascular delivery of siRNA inhibits EGFP expression in the liver of transgenic mice. EGFP (green), phalloidin (red). 10 week old mice (strain C57BL/6-TgN(ACTbEGFP)10sb) expressing EGFP were injected with 50 μg siRNA (mice #1 and 2), 50 μg control siRNA (mice #3 and 4) or were not injected (mouse #5). Livers were harvested 30 h post-injection, sectioned, fixed, and counterstained with Alexa 568 phalloidin in order to visualize cell outlines. Images were acquired using a Zeiss Axioplan fluorescence microscope outfitted with a Zeiss AxioCam digital camera.

FIG. 3. A) Delivery of siRNA-Luc+. Maximal inhibition is achieved at 10 nM siRNA-Luc+. B) Delivery of morpholino-Luc+. Maximal specific inhibition is achieved at 100 nM morpholino-Luc+. C) Comparison of inhibitory power of siRNA-Luc+(1.0 nM) alone, morpholino-Luc+ (100 nM) alone and siRNA-Luc+ (1.0 nM) plus morpholino (100 nM) together. When siRNA and morpholino are added together at these concentrations, the degree of inhibition is greater than either siRNA or morphlino alone. D) Comparison of inhibitory power of siRNA-Luc+ (10 nM) alone, morpholino- Luc+ (100 nM) alone and siRNA-Luc+ (10 nM) plus morpholino (100 nM) together. When siRNA and morpholino are added together at these concentrations, the degree of inhibition is greater than either siRNA or morphlino alone.

FIG. 4. Peak gene transfer activities of DNA/brPEI/polyanion complexes applied to HUH7 cells in 100% bovine serum. The peak activities were obtained in titration experiments. pAA, polyacrylic acid; pAsp, polyaspartic acid; pGlu, polyglutamic acid; SPLL, succinylated poly-L-lysine.

FIG. 5. Gene transfer activity of DNA/lPEI/pAA complexes in lungs at different amounts of pAA added to DNA/lPEI (50 μg/200 μg) and intravenously injected in mice. The complexes were formulated in isotonic glucose. Each experimental point is an average out of two animals.

FIG. 6. A) ALT blood levels for animals systemically injected with DNA/lPEI/pAA complex alone (50 μg/400 μg/50 μg) or complex followed by pAA “chaser” tail vein injection 30 min later. B) Luciferase expression levels in lung followed systemic injections of DNA/lPEI/pAA complex alone and the complex/“chaser” combination.

### SUMMARY

In a preferred embodiment we describe processes for delivering a RNA function inhibitor (hereafter referred to as “inhibitor”) to an animal cell. We also describe compositions that facilitate delivery of an inhibitor to an animal cell. Delivery of the inhibitor results in inhibition of target gene expression by causing degradation of inhibition of function of RNA. Inhibitors are selected from the group comprising siRNA, dsRNA, antisense nucleic acid, ribozymes, RNA polymerase III transcribed DNAs, and the like. A preferred inhibitor is siRNA.

In a preferred embodiment, we describe an in vivo process for delivery of an inhibitor to a cell of a mammal for the purposes of inhibition of gene expression (RNA function) comprising making an inhibitor, injecting the inhibitor into a vessel, and delivering the inhibitor to a cell within a tissue thereby inhibiting expression of a target gene in the cell. Permeability of the vessel to the inhibitor may comprise increasing the pressure within the vessel by rapidly injecting a large volume of fluid into the vessel and blocking the flow of fluid into and/or out of the target tissue. This increased pressure is controlled by altering the injection volume, altering the rate of volume insertion, and by constricting the flow of blood into or out of the tissue during the procedure. The volume consists of an inhibitor in a solution wherein the solution may contain a compound or compounds which may or may not complex with the inhibitor and aid in delivery.

In a preferred embodiment, a process is described for increasing the transit of the inhibitor out of a vessel and into the cells of the surrounding tissue, comprising rapidly injecting a large volume into a vessel supplying the target tissue, thus forcing fluid out of the vasculature into the extravascular space. This process is accomplished by forcing a volume containing the inhibitor into a vessel and either constricting the flow of fluid into and/or out of an area, adding a molecule that increases the permeability of a vessel, or both. The target tissue comprises the cells supplied by the vessel distal to the point of injection. For injection into arteries, the target tissue is the cells that the arteries supply with blood. For injection into veins, the target tissue is the cells from which the vein drains blood.

In a preferred embodiment, an in vivo process for delivering an inhibitor to mammalian cells consists of inserting the inhibitor into a vessel and applying pressure to the vessel proximal to the point of injection and target tissue. The process includes impeding fluid flow into and away from the target tissue through afferent and efferent vessels by externally applying pressure to interior vessels such as by compressing mammalian skin. Compressing mammalian skin includes applying a cuff over the skin, such as a sphygmomanometer or a tourniquet. Fluid flow through vessels may also be constricted by directly clamping the vessels such as by a clamp or a balloon catheter. The vessels are occluded for a period of time necessary to delivery the inhibitor without causing damage to the tissue.

In a preferred embodiment, a complex for delivering an inhibitor to a cell in a mammal is provided comprising, mixing an inhibitor with a compound in solution to form a complex wherein the zeta potential of the complex is less negative than the zeta potential of the inhibitor alone. In another preferred embodiment, the inhibitor is complexed with a compound wherein the zeta potential of the complex is not negative. The compound is selected from the group comprising: polymers, polycations, polyampholytes, amphipathic compounds, non-viral vectors, signal molecules, and peptides. The complex is inserted into a mammalian vessel and the permeability of the vessel may be increased. Delivery of the inhibitor to a cell thereby inhibits expression of the target gene in the cell.

In a preferred embodiment, we describe a process for recharging particle for delivery of an inhibitor to an animal cell in vivo or in vitro. We describe the use of a polyanion or polyampholyte in recharging a inhibitor/cationic lipid (CL) or inhibitor/polycation (PC) complex. The polyanion or polyampholyte, when added to the complex, results in increased inhibitor delivery. By caging the inhibitor in 2 layers (first a cationic layer, second an anionic layer), stable particles are formed. The polyanion layer prevents the siRNA-containing core from opsonization by serum proteins^{9,20,21}. The polyanion outer layer also reduces damage to endothelial cells lining the lungs and/or liver thereby decreasing toxicity. Precise titration of siRNA/polycation complex with polyanion results in a significant increase in gene transfer activity both in vitro and in vivo in a narrow range of polyanion concentrations. Recharging the complex results in increased gene transfer activity and decreased toxicity. The recharged particles are also more stable in physiologic salt and serum. In a preferred embodiment, the components of the complex may be modified to enhance its extracellular or intracellular qualities. For example, endosomolytic function can be enhanced by conjugation of endosomolytic compounds. Molecules that increase cell binding or internalization or enhance cell type specific binding may also be attached to the inhibitor-containing complex. The recharged complex may be used to deliver the inhibitor to cells in the lung. The particles can be delivered intranasally, intratracheally, or intravenously.

In a preferred embodiment, an inhibitor-containing complex is stabilized by using a cross-linking reagent. For instance, in a ternary complex comprising an inhibitor, a polycation, and a polyanion, the polycation may be crosslinked to itself, to the polyanion, or to the inhibitor. The polyanion may also be crosslinked to itself, to the polycation, or to the inhibitor. Crosslinking enhances the stability of the complex.

In a preferred embodiment, ternary complexes comprising an inhibitor, amphipathic compounds and polycations, and processes using such complexes to deliver the inhibitor to an animal cell in vivo or in vitro for the purposes of inhibiting expression of a target gene in the cell are described. The use of a polycation and an amphipathic compound together significantly increase inhibitor transfer efficiency.

Polycations are selected from the group comprising poly-L-lysine, polyethylenimine (PEI), polysilazane, polydihydroimidazolenium, polyallylamine, and proteins. A preferred cationic polymer is ethoxylated polyethylenimine (ePEI). Another preferred polycation is a DNA-binding protein. A preferred DNA-binding protein is a histone such as H1, H2A, or H2B. The histone can be from a natural source such as calf thymus or can be recombinant protein produced in bacteria. In a preferred embodiment, the DNA-binding protein is linked to a nuclear localization signal such as a recombinant histone containing both the SV40 large T antigen nuclear localization signal and the C-terminal domain of human histone H1, (NLS-H1).

A variety of amphipathic compounds can be used in conjunction with a polycation to mediate the transfer of the siRNA into the cell. In a preferred embodiment the amphipathic compound is cationic. The cationic amphipathic compound can be a non-natural polyamine wherein one or more of the amines is bound to at least one hydrophobic moiety selected from the group comprising: C6 to C24 alkane, C6-C24 alkene, cycloalkyl, sterol, steroid, appropriately substituted lipid, acyl segment of a fatty acid, hydrophobic hormone, or other similar hydrophobic group. The amphipathic compounds may or may not form liposomes. A preferred, amphipathic cationic compound has the general structure comprising:

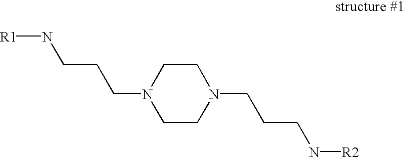

wherein R₁ and R₂ are hydrophobic moieties selected from the group comprising: C6 to C24 alkane, C6-C24 alkene, cycloalkyl, sterol, steroid, appropriately substituted lipid, acyl segment of a fatty acid, hydrophobic hormone, or other similar hydrophobic group. R₁ may be identical to R₂ or R₁ may be different from R₂. The combination of polycation and amphipathic compounds enhances the efficiency of inhibitor transfer into a variety of animal cells with minimal cellular toxicity.

In a preferred embodiment, polyethylenimine or a similar polymer is used as the polycation and a compound of structure #1 is used as the amphipathic compound. In another preferred embodiment, histone H1 protein is used as the polycation and a compound of structure #1 is used as the amphipathic compound. In another preferred embodiment, these amphipathic compounds may be combined with other amphipathic compounds, such a lipids, to form liposomes which are then used to delivery an inhibitor to an animal cell.

In a preferred embodiment the compound, compositions, and processes for delivery of an inhibitor to an animal cell can be used wherein the cell is located in vitro, ex vivo, in situ, or in vivo. The cell can be an animal cell that is maintained in tissue culture such as cell lines that are immortalized or transformed. The cell can be a primary or secondary cell which means that the cell has been maintained in culture for a relatively short time after being obtained from an animal. The cell can also be a mammalian cell that is within the tissue in situ or in vivo meaning that the cell has not been removed from the tissue or the animal.

In a preferred embodiment, the present invention provides a process for delivering an inhibitor to an animal cell comprising; preparing a ternary complex comprising mixing an amphipathic compound with an inhibitor and an effective amount of a polycation in a solution, associating the complex with an animal cell, and delivering the inhibitor to the interior of the cell. The inhibitor then inhibits expression of a gene in the cell. The amphipathic compound may be mixed with the polycation prior to addition of the inhibitor, at the same time as the inhibitor, or after the inhibitor. The term deliver means that the siRNA becomes associated with the cell thereby altering the endogenous properties of the cell by inhibiting expression of a gene. Other terms sometimes used interchangeably with deliver include transfect, transfer, or transform.

In a preferred embodiment, the polycation, the siRNA, the polyanion or the amphipathic compound may be modified by attachment of a functional group. The functional group can be, but is not limited to, a targeting signal or a label or other group that facilitates delivery of the inhibitor. The group may be attached to one or more of the components prior to complex formation. Alternatively, the group(s) may be attached to the complex after formation of the complex.

In a preferred embodiment, a combination of two or more inhibitors are delivered together or sequentially to enhance inhibition of target gene expression. The inhibitors comprise sequence that is identical, nearly identical, or complementary to the same, different, or overlapping segments of the target gene sequence(s). For instance, a preferred combination comprises one inhibitor that is a siRNA and another inhibitor that is an antisense polynucleotide. A preferred antisense polynucleotide is a morpholino or a 2′-O-methyl oligonucleotide. The inhibitors may be delivered to cells in vivo, ex vivo, in situ, or in vitro.

In a preferred embodiment, an inhibitor may be delivered to a cell in a mammal for the purposes of inhibiting a target gene to provide a therapeutic effect. The target gene is selected from the group that comprises: dysfunctional endogenous genes and viral or other infectious agent genes. Dysfunctional endogenous genes include dominant genes which cause disease and cancer genes. Genes that express proteins that can be converted to prions are another potential target of an inhibitor. The inhibitor is delivered to reduce expression of the prion gene thereby lessening abnormal plaque formation caused by the altered gene product.

In a preferred embodiment, an inhibitor is delivered to a mammalian cell in vivo for the treatment of a disease or infection. The inhibitor reduces expression of a viral or bacterial gene. The inhibitor may reduce or block microbe production, virulence, or both. Delivery of the inhibitor may delay progression of disease until endogenous immune protection can be acquired. Viral genes involved in transcription, replication, virion assembly, immature viral membrane formation, extracellular enveloped virus formation, early genes, intermediate genes, late genes, and virulence genes may be targeted. Bacterial genes involved in transcription, replication, virulence, cell growth, pathogenicity, etc. may be targeted. In a preferred embodiment, combinations of effective inhibitors targeted to the same or different viral genes or classes of genes (e.g., transcription, replication, virulence, etc) are delivered to an infected mammalian cell in vivo. Examples of infectious agents that may be treated in this manner include biological warfare agents such as the small pox virus and Anthrax.

Alternatively, instead of inhibiting an infectious agent gene, the inhibitor may decrease expression of an endogenous host gene to reduce virulence of the pathogen. The inhibitor may be delivered to a cell in a mammal to reduce expression of a cellular receptor. For example, the lethality of Anthrax is primarily mediated by a secreted tripartite toxin which requires the mammalian anthrax toxin receptor (ATR) for cellular entry²². Reducing expression of ATR may decrease Anthrax toxicity. Receptors to which pathogens bind may also be targeted.

In a preferred embodiment, an inhibitor is delivered to a mammalian cell in vivo to modulate immune response. Since host immune response is responsible for the toxicity of some infectious agents, reducing this response may increase the survival of an infected mammal. Also, inhibition of immune response is beneficial for a number of other therapeutic purposes, including gene therapy, where immune reaction often greatly limits transgene expression, organ transplantation, and autoimmune disorders.

In a preferred embodiment, an inhibitor is delivered to a mammalian cell for the purpose of facilitating pharmaceutical drug discovery or target validation. The mammalian cell may be in vitro or in vivo. Specific inhibition of a target gene can aid in determining whether an inhibition of a protein or gene has a significant phenotypic effect. Specific inhibition of a target gene can also be used to study the target gene's effect on the cell.

Further objects, features, and advantages of the invention will be apparent from the following detailed description when taken in conjunction with the accompanying drawings.

### DETAILED DESCRIPTION

We have found that an intravascular route of administration allows an RNA function inhibitor (inhibitor) to be delivered to a mammalian cell in a more even distribution than direct parenchymal injections. The efficiency of inhibitor delivery may be increased by increasing the permeability of the tissue's blood vessel. Permeability is increased by increasing the intravascular hydrostatic pressure (above, for example, the resting diastolic blood pressure in a blood vessel), delivering the injection fluid rapidly (injecting the injection fluid rapidly), using a large injection volume, and/or increasing permeability of the vessel wall.

RNA Function Inhibitor

A RNA function inhibitor (“inhibitor”) comprises any nucleic acid or nucleic acid analog containing a sequence (“inhibiting sequence”) whose presence or expression in a cell causes the degradation of or inhibits the function or translation of a specific cellular RNA, usually a mRNA, in a sequence-specific manner. Inhibition of RNA can thus effectively inhibit expression of a gene from which the RNA is transcribed. Inhibitors are selected from the group comprising: siRNA, interfering RNA or RNAi, dsRNA, RNA Polymerase III transcribed DNAs, ribozymes, and antisense nucleic acid, which may be RNA, DNA, or artificial nucleic acid. SiRNA comprises a double stranded structure typically containing 15-50 base pairs and preferably 21-25 base pairs and having a nucleotide sequence identical or nearly identical to an expressed target gene or RNA within the cell. Antisense polynucleotides include, but are not limited to: morpholinos, 2′-O-methyl polynucleotides, DNA, RNA and the like. RNA polymerase III transcribed DNAs contain promoters, such as the U6 promoter. These DNAs can be transcribed to produce small hairpin RNAs in the cell that can function as siRNA²³ or linear RNAs that can function as antisense RNA. The inhibitor may be polymerized in vitro, recombinant RNA, contain chimeric sequences, or derivatives of these groups. The inhibitor may contain ribonucleotides, deoxyribonucleotides, synthetic nucleotides, or any suitable combination such that the target RNA and/or gene is inhibited. In addition, these forms of nucleic acid may be single, double, triple, or quadruple stranded.

A delivered inhibitor can stay within the cytoplasm or nucleus. The inhibitor can be delivered to a cell to inhibit expression of an endogenous or exogenous nucleotide sequence or to affect a specific physiological characteristic not naturally associated with the cell.

An inhibitor can be delivered to a cell in order to produce a cellular change that is therapeutic. The delivery of an inhibitor or other genetic material for therapeutic purposes (the art of improving health in an animal including treatment or prevention of disease) is called gene therapy. The inhibitor can be delivered either directly to the organism in situ or indirectly by transfer to a cell ex vivo that is then transplanted into the organism. Entry into the cell is required for the inhibitor to block the production of a protein or to decrease the amount of a target RNA. Diseases, such as autosomal dominant muscular dystrophies, which are caused by dominant mutant genes, are examples of candidates for treatment with therapeutic inhibitors such as siRNA. Delivery of the inhibitor would block production of the dominant protein without affecting the normal protein thereby lessening the disease.

We demonstrate that delivery of siRNA and antisense inhibitors to cells of post-embryonic mice and rats interferes with specific gene expression in those cells. The inhibition is gene specific and does not cause general translational arrest. Thus RNAI can be effective in post-embryonic mammalian cells in vivo.

Nucleic Acid/Polynucleotide

The term nucleic acid, or polynucleotide, is a term of art that refers to a string of at least two nucleotides. Nucleotides are the monomeric units of nucleic acid polymers. Polynucleotides with less than 120 monomeric units are often called oligonucleotides. Natural nucleic acids have a deoxyribose-or ribose-phosphate backbone while artificial polynucleotides are polymerized in vitro and contain the same or similar bases but may contain other types of backbones. These backbones include: PNAs (peptide nucleic acids), phosphorothioates, phosphorodiamidates, morpholinos, and other variants of the phosphate backbone of native nucleic acids. Bases include purines and pyrimidines, which further include the natural compounds adenine, thymine, guanine, cytosine, uracil, inosine, and natural analogs. Synthetic derivatives of purines and pyrimidines include, but are not limited to, modifications which place new reactive groups on the base such as, but not limited to, amines, alcohols, thiols, carboxylates, and alkylhalides. The term base encompasses any of the known base analogs of DNA and RNA including, but not limited to: 4-acetylcytosine, 8-hydroxy-N6-methyladenosine, aziridinylcytosine, pseudoisocytosine, 5-(carboxyhydroxyl-methyl) uracil, 5-fluorouracil, 5-bromouracil, 5-carboxymethylaminomethyl-2-thiouracil, 5-carboxymethyl-aminomethyluracil, dihydrouracil, inosine, N6-isopentenyladenine, 1-methyladenine, 1-methylpseudo-uracil, 1-methylguanine, 1-methylinosine, 2,2-dimethyl-guanine, 2-methyladenine, 2-methylguanine, 3-methyl-cytosine, 5-methylcytosine, N6-methyladenine, 7-methylguanine, 5-methylaminomethyluracil, 5-methoxy-amino-methyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine. The term includes deoxyribonucleic acid (DNA) and ribonucleic acid (RNA). DNA may be in form of cDNA, in vitro polymerized DNA, plasmid DNA, parts of a plasmid DNA, genetic material derived from a virus, linear DNA, chromosomal DNA, an oligonucleotide, antisense DNA, or derivatives of these groups. RNA may be in the form of tRNA (transfer RNA), snRNA (small nuclear RNA), rRNA (ribosomal RNA), mRNA (messenger RNA), antisense RNA, siRNA (small interfering RNA), dsRNA (double stranded RNA), RNAi, ribozymes, in vitro polymerized RNA, or derivatives of these groups.

Deliver

The term deliver means that the inhibitor becomes associated with the cell thereby altering the properties of the cell by inhibiting function of an RNA The inhibitor can be on the membrane of the cell or inside the cytoplasm, nucleus, or other organelle of the cell. Other terms sometimes used interchangeably with deliver include transfect, transfer, or transform. In vivo delivery of an inhibitor means to transfer the inhibitor from a container outside a mammal to near or within the outer cell membrane of a cell in the mammal. The inhibitor can interfere with RNA function in either the nucleus or cytoplasm.

Cells

Using the described invention, inhibitors are efficiently delivered to cells in culture, i.e., in vitro. These include a number of cell lines that can be obtained from American Type Culture Collection (Bethesda) such as, but not limited to: 3T3 (mouse fibroblast) cells, Rat1 (rat fibroblast) cells, CHO (Chinese hamster ovary) cells, CV-1 (monkey kidney) cells, COS (monkey kidney) cells, 293 (human embryonic kidney) cells, HeLa (human cervical carcinoma) cells, HepG2 (human hepatocytes) cells, Sf9 (insect ovarian epithelial) cells and the like.

The invention also describes the delivery of an inhibitor to a cell that is in vivo, in situ, ex vivo or a primary cell. Primary cells include, but are not limited to, primary liver cells and primary muscle cells and the like. For primary cells, the cells within the tissue are separated by mincing and digestion with enzymes such as trypsin or collagenases which destroy the extracellular matrix. Tissues consist of several different cell types. Purification methods such as gradient centrifugation or antibody sorting can be used to obtain purified amounts of the preferred cell type. For example, primary myoblasts are separated from contaminating fibroblasts using Percoll (Sigma) gradient centrifugation.

Parenchymal Cells

Parenchymal cells are the distinguishing cells of a gland or organ contained in and supported by the connective tissue framework. The parenchymal cells typically perform a function that is unique to the particular organ. The term “parenchymal” often excludes cells that are common to many organs and tissues such as fibroblasts and endothelial cells within blood vessels.

For example, in a liver organ, the parenchymal cells include hepatocytes, Kupffer cells and the epithelial cells that line the biliary tract and bile ductules. The major constituent of the liver parenchyma are polyhedral hepatocytes (also known as hepatic cells) that presents at least one side to an hepatic sinusoid and opposed sides to a bile canaliculus. Liver cells that are not parenchymal cells include cells within the blood vessels such as the endothelial cells or fibroblast cells. In one preferred embodiment hepatocytes are targeted by injecting the inhibitor or inhibitor complex into the portal vein or bile duct of a mammal.

In striated muscle, the parenchymal cells include myoblasts, satellite cells, myotubules, and myofibers. In cardiac muscle, the parenchymal cells include the myocardium also known as cardiac muscle fibers or cardiac muscle cells and the cells of the impulse connecting system such as those that constitute the sinoatrial node, atrioventricular node, and atrioventricular bundle.

Vessel

Vessels comprise internal hollow tubular structures connected to a tissue or organ within the body. Bodily fluid flows to or from the body part within the cavity of the tubular structure. Examples of bodily fluid include blood, lymphatic fluid, or bile. Examples of vessels include arteries, arterioles, capillaries, venules, sinusoids, veins, lymphatics, and bile ducts. Afferent blood vessels of organs are defined as vessels which are directed towards the organ or tissue and in which blood flows towards the organ or tissue under normal physiological conditions. Conversely, efferent blood vessels of organs are defined as vessels which are directed away from the organ or tissue and in which blood flows away from the organ or tissue under normal physiological conditions. In the liver, the hepatic vein is an efferent blood vessel since it normally carries blood away from the liver into the inferior vena cava. Also in the liver, the portal vein and hepatic arteries are afferent blood vessels in relation to the liver since they normally carry blood towards the liver. Insertion of the inhibitor or inhibitor complex into a vessel enables the inhibitor to be delivered to parenchymal cells more efficiently and in a more even distribution compared with direct parenchymal injections.

Increasing Vessel Permeability

In a preferred embodiment, the permeability of the vessel is increased. Efficiency of inhibitor delivery is increased by increasing the permeability of a vessel within the target tissue. Permeability is defined here as the propensity for macromolecules such as an inhibitor to exit the vessel and enter extravascular space. One measure of permeability is the rate at which macromolecules move out of the vessel. Another measure of permeability is the lack of force that resists the movement of inhibitors being delivered to leave the intravascular space.

Rapid injection may be combined with obstructing the outflow to increase permeability. To obstruct, in this specification, is to block or inhibit inflow or outflow of fluid through a vessel. For example, an afferent vessel supplying an organ is rapidly injected and the efferent vessel draining the tissue is ligated transiently. The efferent vessel (also called the venous outflow or tract) draining outflow from the tissue is also partially or totally clamped for a period of time sufficient to allow delivery of a polynucleotide. In the reverse, an efferent is injected and an afferent vessel is occluded.

In another preferred embodiment, the pressure of a vessel is increased by increasing the osmotic pressure within the vessel. Typically, hypertonic solutions containing salts such as NaCl, sugars or polyols such as mannitol are used. Hypertonic means that the osmolarity of the injection solution is greater than physiological osmolarity. Isotonic means that the osmolarity of the injection solution is the same as the physiological osmolarity (the tonicity or osmotic pressure of the solution is similar to that of blood). Hypertonic solutions have increased tonicity and osmotic pressure relative to the osmotic pressure of blood and cause cells to shrink.

In another preferred embodiment, the permeability of a vessel can be increased by a biologically-active molecule. A biologically-active molecule is a protein or a simple chemical such as papaverine or histamine that increases the permeability of the vessel by causing a change in function, activity, or shape of cells within the vessel wall such as the endothelial or smooth muscle cells. Typically, biologically-active molecules interact with a specific receptor or enzyme or protein within the vascular cell to change the vessel's permeability. Biologically-active molecules include vascular permeability factor (VPF) which is also known as vascular endothelial growth factor (VEGF). Another type of biologically-active molecule can increase permeability by changing the extracellular connective material. For example, an enzyme could digest the extracellular material and increase the number and size of the holes of the connective material.

In a preferred embodiment, an inhibitor or inhibitor-containing complex is injected into a vessel in a large injection volume. The injection volume is dependent on the size of the animal to be injected and can be from 1.0 to 3.0 ml or greater for small animals (i.e. tail vein injections into mice). The injection volume for rats can be from 6 to 35 ml or greater. The injection volume for primates can be 70 to 200 ml or greater. The injection volumes in terms of ml/body weight can be 0.03 ml/g to 0.1 ml/g or greater.

The injection volume can also be related to the target tissue. For example, delivery of a non-viral vector with an inhibitor to a limb can be aided by injecting a volume greater than 5 ml per rat limb or greater than 70 ml for a primate. The injection volumes in terms of ml/limb muscle are usually within the range of 0.6 to 1.8 ml/g of muscle but can be greater. In another example, delivery of an inhibitor or inhibitor complex to liver in mice can be aided by injecting the inhibitor in an injection volume from 0.6 to 1.8 ml/g of liver or greater. In another example delivering an inhibitor to a limb of a primate (rhesus monkey), the inhibitor or complex can be in an injection volume from 0.6 to 1.8 ml/g of limb muscle or anywhere within this range.

In another embodiment the injection fluid is injected into a vessel rapidly. The speed of the injection is partially dependent on the volume to be injected, the size of the vessel into which the volume is injected, and the size of the animal. In one embodiment the total injection volume (1-3 ml) can be injected from 15 to 5 seconds into the vascular system of mice. In another embodiment the total injection volume (6-35 ml) can be injected into the vascular system of rats from 20 to 7 seconds. In another embodiment the total injection volume (80-200 ml) can be injected into the vascular system of monkeys from 120 seconds or less.

In another embodiment a large injection volume is used and the rate of injection is varied. Injection rates of less than 0.012 ml per gram (animal weight) per second are used in this embodiment. In another embodiment injection rates of less than 0.2 ml per gram (target tissue weight) per second are used for gene delivery to target organs. In another embodiment injection rates of less than 0.06 ml per gram (target tissue weight) per second are used for gene delivery into limb muscle and other muscles of primates.

Polymer

A polymer is a molecule built up by repetitive bonding together of smaller units called monomers. Small polymer having 2 to about 80 monomers can be called oligomers. The polymer can be linear, branched network, star, comb, or ladder type. The polymer can be a homopolymer in which a single monomer is used or can be copolymer in which two or more monomers are used. Types of copolymers include alternating, random, block and graft.

The main chain of a polymer is composed of the atoms whose bonds are required for propagation of polymer length. The side chain of a polymer is composed of the atoms whose bonds are not required for propagation of polymer length.

To those skilled in the art, there are several categories of polymerization processes that can be utilized in the described process. The polymerization can be chain or step. This classification description is more often used than the previous terminology of addition and condensation polymerization. “Most step-reaction polymerizations are condensation processes and most chain-reaction polymerizations are addition processes” (M. P. Stevens Polymer Chemistry: An Introduction New York Oxford University Press 1990). Template polymerization can be used to form polymers from daughter polymers.

Step Polymerization

In step polymerization, the polymerization occurs in a stepwise fashion. Polymer growth occurs by reaction between monomers, oligomers and polymers. No initiator is needed since the same reaction occurs throughout and there is no termination step so that the end groups are still reactive. The polymerization rate decreases as the functional groups are consumed.

Typically, step polymerization is done either of two different ways. One way, the monomer has both reactive functional groups (A and B) in the same molecule so that

A—B yields —[A—B—]—

The other approach is to have two difunctional monomers.

A—A+B—B yields —[A—A—B—B]—

Generally, these reactions can involve acylation or alkylation. Acylation is defined as the introduction of an acyl group (—COR) onto a molecule. Alkylation is defined as the introduction of an alkyl group onto a molecule.

If functional group A is an amine then B can be (but not restricted to) an isothiocyanate, isocyanate, acyl azide, N-hydroxysuccinimide, sulfonyl chloride, aldehyde (including formaldehyde and glutaraldehyde), ketone, epoxide, carbonate, imidoester, carboxylate, or alkylphosphate, arylhalides (difluoro-dinitrobenzene), anhydrides or acid halides, p-nitrophenyl esters, o-nitrophenyl pentachlorophenyl esters, or pentafluorophenyl esters. In other terms when function A is an amine then function B can be acylating or alkylating agent or amination.

If functional group A is a thiol (sulfhydryl) then function B can be (but not restricted to) an iodoacetyl derivative, maleimide, aziridine derivative, acryloyl derivative, fluorobenzene derivatives, or disulfide derivative (such as a pyridyl disulfide or 5-thio-2-nitrobenzoic acid (TNB) derivatives).

If functional group A is carboxylate then function B can be (but not restricted to) a diazoacetate or an amine in which a carbodiimide is used. Other additives may be utilized such as carbonyldiimidazole, dimethylaminopyridine, N-hydroxysuccinimide or alcohol using carbodiimide and dimethylaminopyridine.

If functional group A is a hydroxyl then function B can be (but not restricted to) an epoxide, oxirane, or an amine in which carbonyldiimidazole or N,N′-disuccinimidyl carbonate, or N-hydroxysuccinimidyl chloroformate or other chloroformates are used.

If functional group A is an aldehyde or ketone then function B can be (but not restricted to) an hydrazine, hydrazide derivative, amine (to form a imine or iminium that may or may not be reduced by reducing agents such as NaCNBH₃) or hydroxyl compound to form a ketal or acetal.

Yet another approach is to have one difunctional monomer so that

A—A plus another agent yields —[A—A]—.

If function A is a thiol group then it can be converted to disulfide bonds by oxidizing agents such as iodine (I₂) or NaIO₄ (sodium periodate), or oxygen (O₂). Function A can also be an amine that is converted to a thiol group by reaction with 2-Iminothiolate (Traut's reagent), which then undergoes oxidation and disulfide formation. Disulfide derivatives (such as a pyridyl disulfide or TNB derivatives) can also be used to catalyze disulfide bond formation.

Functional group A or B in any of the above examples could also be a photoreactive group such as aryl azides, halogenated aryl azides, diazo, benzophenones, alkynes or diazirine derivatives.

Reactions of the amine, hydroxyl, thiol carboxylate groups yield chemical bonds that are described as amide, amidine, disulfide, ethers, esters, enamine, urea, isothiourea, isourea, sulfonamide, carbamate, carbon-nitrogen double bond (imine), alkylamine bond (secondary amine), carbon-nitrogen single bonds in which the carbon contains a hydroxyl group, thio-ether, diol, hydrazone, diazo, or sulfone.

Chain Polymerization

In chain-reaction polymerization growth of the polymer occurs by successive addition of monomer units to limited number of growing chains. The initiation and propagation mechanisms are different and there is usually a chain-terminating step. The polymerization rate remains constant until the monomer is depleted.

Monomers containing vinyl, acrylate, methacrylate, acrylamide, methacrylamide groups can undergo chain reaction, which can be radical, anionic, or cationic. Chain polymerization can also be accomplished by cycle or ring opening polymerization. Several different types of free radical initiators can be used that include peroxides, hydroxy peroxides, and azo compounds such as 2,2′-Azobis(-amidinopropane) dihydrochloride (AAP).

Monomers

A wide variety of monomers can be used in the polymerization processes. These include positive charged organic monomers such as amines, imidine, guanidine, imine, hydroxylamine, hydrazine, heterocycles (like imidazole, pyridine, morpholine, pyrimidine, or pyrene). The amines can be pH-sensitive in that the pKₐ of the amine is within the physiologic range of 4 to 8. Specific amines include spermine, spermidine, N,N′-bis(2-aminoethyl)-1,3-propanediamine (AEPD), and 3,3′-Dianino-N,N-dimethyldipropylammonium bromide.

Monomers can also be hydrophobic, hydrophilic or amphipathic. Monomers can also be intercalating agents such as acridine, thiazole organge, or ethidium bromide.

The polymers can also contain cleavable groups either in the main chain or in side chains. Cleavable groups include but are not restricted to disulfide bonds, diols, diazo bonds, ester bonds, sulfone bonds, acetals, ketals, enol ethers, enol esters, enamines and imines. Preferred cleavable groups include groups that are pH labile.

The polymers may have other functional groups or modifications that increase their utility. These groups can be incorporated into monomers prior to polymer formation or attached to the polymer after its formation.

Polyelectrolyte/Polycation/Polyanion

A polyelectrolyte, or polyion, is a polymer possessing more than one charge, i.e. the polymer contains groups that have either gained or lost one or more electrons. A polycation is a polyelectrolyte possessing net positive charge, for example poly-L-lysine hydrobromide. The polycation can contain monomer units that are charge positive, charge neutral, or charge negative, however, the net charge of the polymer must be positive. A polycation also can mean a non-polymeric molecule that contains two or more positive charges. A polyanion is a polyelectrolyte containing a net negative charge. The polyanion can contain monomer units that are charge negative, charge neutral, or charge positive, however, the net charge on the polymer must be negative. A polyanion can also mean a non-polymeric molecule that contains two or more negative charges. The term polyelectrolyte includes polycation, polyanion, zwitterionic polymers, and neutral polymers.

The term zwitterionic refers to the product (salt) of the reaction between an acidic group and a basic group that are part of the same molecule.

Polymers have been used in research for the delivery of nucleic acids to cells. One of the several methods of nucleic acid delivery to the cells is the use of nucleic acid/polycation complexes. It has been shown that cationic proteins, like histones and protamines, or synthetic polymers, like polylysine, polyarginine, polyornithine, DEAE dextran, polybrene, and polyethylenimine, but not small polycations like spermine may be effective intracellular DNA delivery agents. Multivalent cations with a charge of three or higher have been shown to condense nucleic acid when 90% or more of the charges along the sugar-phosphate backbone are neutralized. The volume which one polynucleotide molecule occupies in a complex with polycations is lower than the volume of a free polynucleotide molecule. Polycations also provide attachment of polynucleotide to a cell surface. The polymer forms a cross-bridge between the polyanionic nucleic acid and the polyanionic surface of the cell. As a result, the mechanism of nucleic acid translocation to the intracellular space might be non-specific adsorptive endocytosis. Furthermore, polycations provide a convenient linker for attaching specific ligands to the complex. The nucleic acid/polycation complexes could then be targeted to specific cell types. Complex formation also protects against nucleic acid degradation by nucleases present in serum as well as in endosomes and lysosomes. Protection from degradation in endosomes/lysosomes is enhanced by preventing organelle acidification. Disruption of endosomal/lysosomal function may also be accomplished by linking endosomal or membrane disruptive agents to the polycation or complex.

A DNA-binding protein is a protein that associates with nucleic acid under conditions described in this application and forms a complex with nucleic acid with a high binding constant. The DNA-binding protein can be used in an effective amount in its natural form or a modified form for this process. An “effective amount” of the polycation is an amount that will allow delivery of the inhibitor to occur.

A non-viral vector is defined as a vector that is not assembled within an eukaryotic cell including non- viral inhibitor/polymer complexes, inhibitor with transfection enhancing compounds and inhibitor+amphipathic compounds.

Surface Charging/Recharging

While positive surface charge may facilitate interaction between the nucleic acid/polycation complex and a cell, a negative surface charge would be more desirable for many practical applications, in particular in vivo delivery. The phenomenon of surface recharging is well known in colloid chemistry and is described in great detail for lyophobic/lyophilic systems (i.e., silver halide hydrosols). Addition of polyion to a suspension of latex particles with an oppositely-charged surface leads to the permanent absorption of the polyion onto the surface. Upon reaching appropriate stoichiometry, the surface is changed to the opposite charge.

Zeta potential is the difference in electrical potential between a tightly bound layer of ions on particle surfaces and the liquid in which the particles are suspended.

Amphipathic Compound

An amphipathic compound is a molecule that contains one end that is hydrophilic while the other end is hydrophobic. The term hydrophobic indicates in qualitative terms that the chemical moiety is water-avoiding. Hydrocarbons are hydrophobic groups. The term hydrophilic indicates in qualitative terms that the chemical moiety is water-preferring. Typically, such chemical groups are water soluble, and are hydrogen bond donors or acceptors with water. Examples of hydrophilic groups include compounds with the following chemical moieties; carbohydrates, polyoxyethylene, oligonucleotides, and groups containing amines, amides, alkoxy amides, carboxylic acids, sulfurs, or hydroxyls.

Lipid

A lipid is any of a diverse group of organic compounds that are insoluble in water, but soluble in organic solvents such as chloroform and benzene. Lipids contain both hydrophobic and hydrophilic sections. Lipids is meant to include complex lipids, simple lipids, and synthetic lipids. Complex lipids are the esters of fatty acids and include glycerides (fats and oils), glycolipids, phospholipids, and waxes. Simple lipids include steroids and terpenes. Synthetic lipids includes amides prepared from fatty acids wherein the carboxylic acid has been converted to the amide, synthetic variants of complex lipids in which one or more oxygen atoms has been substituted by another heteroatom (such as Nitrogen or Sulfur), and derivatives of simple lipids in which additional hydrophilic groups have been chemically attached. Synthetic lipids may contain one or more labile groups.

Complex

Two molecules are combined, to form a complex through a process called complexation or complex formation, if the are in contact with one another through noncovalent interactions such as electrostatic interactions, hydrogen bonding interactions, and hydrophobic interactions. An interpolyelectrolyte complex is a noncovalent interaction between polyelectrolytes of opposite charge.

Modification

A molecule is modified, to form a modification through a process called modification, by a second molecule if the two become bonded through a covalent bond. That is, the two molecules form a covalent bond between an atom from one molecule and an atom from the second molecule resulting in the formation of a new single molecule. A chemical covalent bond is an interaction, bond, between two atoms in which there is a sharing of electron density. Modification also means an interaction between two molecules through a noncovalent bond. For example crown ethers can form noncovalent bonds with certain amine groups.

Functional Group

Functional groups include cell targeting signals, nuclear localization signals, compounds that enhance release of contents from endosomes or other intracellular vesicles (releasing signals), and other compounds that alter the behavior or interactions of the compound are complex to which they are attached.

Cell targeting signals are any signals that enhance the association of the biologically active compound with a cell. These signals can modify a biologically active compound such as drug or nucleic acid and can direct it to a cell location (such as tissue) or location in a cell (such as the nucleus) either in culture or in a whole organism. The signal may increase binding of the compound to the cell surface and/or its association with an intracellular compartment. By modifying the cellular or tissue location of the foreign gene, the function of the biologically active compound can be enhanced. The cell targeting signal can be, but is not limited to, a protein, peptide, lipid, steroid, sugar, carbohydrate, (non-expressing) polynucleic acid or synthetic compound. Cell targeting signals such as ligands enhance cellular binding to receptors. A variety of ligands have been used to target drugs and genes to cells and to specific cellular receptors. The ligand may seek a target within the cell membrane, on the cell membrane or near a cell. Binding of ligands to receptors typically initiates endocytosis. Ligands include agents that target to the asialoglycoprotein receptor by using asiologlycoproteins or galactose residues. Other proteins such as insulin, EGF, or transferrin can be used for targeting. Peptides that include the RGD sequence can be used to target many cells. Chemical groups that react with thiol, sulfhydryl, or disulfide groups on cells can also be used to target many types of cells. Folate and other vitamins can also be used for targeting. Other targeting groups include molecules that interact with membranes such as lipids, fatty acids, cholesterol, dansyl compounds, and amphotericin derivatives. In addition viral proteins could be used to bind cells.

After interaction of the supramolecular complexes with the cell, other targeting groups can be used to increase the delivery of the drug or nucleic acid to certain parts of the cell. For example, agents can be used to disrupt endosomes and a nuclear localizing signal (NLS) can be used to target the nucleus.

Nuclear localizing signals enhance the targeting of the pharmaceutical into proximity of the nucleus and/or its entry into the nucleus. Such nuclear transport signals can be a protein or a peptide such as the SV40 large T antigen NLS or the nucleoplasmin NLS. These nuclear localizing signals interact with a variety of nuclear transport factors such as the NLS receptor (karyopherin alpha) which then interacts with karyopherin beta. The nuclear transport proteins themselves could also function as NLS's since they are targeted to the nuclear pore and nucleus. For example, karyopherin beta itself could target the DNA to the nuclear pore complex. Several peptides have been derived from the SV40 T antigen. Other NLS peptides have been derived from M9 protein, nucleoplasmin, and c-myc.

Many biologically active compounds, in particular large and/or charged compounds, are incapable of crossing biological membranes. In order for these compounds to enter cells, the cells must either take them up by endocytosis, i.e., into endosomes, or there must be a disruption of the cellular membrane to allow the compound to cross. In the case of endosomal entry, the endosomal membrane must be disrupted to allow for movement out of the endosome and into the cytoplasm. Either entry pathway into the cell requires a disruption of the cellular membrane. Compounds that disrupt membranes or promote membrane fusion are called membrane active compounds. These membrane active compounds, or releasing signals, enhance release of endocytosed material from intracellular compartments such as endosomes (early and late), lysosomes, phagosomes, vesicle, endoplasmic reticulum, golgi apparatus, trans golgi network (TGN), and sarcoplasmic reticulum. Release includes movement out of an intracellular compartment into the cytoplasm or into an organelle such as the nucleus. Releasing signals include chemicals such as chloroquine, bafilomycin or Brefeldin A1 and the ER-retaining signal (KDEL sequence), viral components such as influenza virus hemagglutinin subunit HA-2 peptides and other types of amphipathic peptides. The control of when and where the membrane active compound is active is crucial to effective transport. If the membrane active agent is operative in a certain time and place it would facilitate the transport of the biologically active compound across the biological membrane. If the membrane active compound is too active or active at the wrong time, then no transport occurs or transport is associated with cell rupture and cell death. Nature has evolved various strategies to allow for membrane transport of biologically active compounds including membrane fusion and the use membrane active compounds whose activity is modulated such that activity assists transport without toxicity. Many lipid-based transport formulations rely on membrane fusion and some membrane active peptides' activities are modulated by pH. In particular, viral coat proteins are often pH-sensitive, inactive at neutral or basic pH and active under the acidic conditions found in the endosome.

Another functional group comprises compounds, such as polyethylene glycol, that decrease interactions between molecules and themselves and with other molecules. Such groups are useful in limited interaction such as between serum factors and the molecule or complex to be delivered.

Labile Bond

A labile bond is a covalent bond that is capable of being selectively broken. That is, the labile bond may be broken in the presence of other covalent bonds without the breakage of other covalent bonds. For example, a disulfide bond is capable of being broken in the presence of thiols without cleavage of any other bonds, such as carbon-carbon, carbon-oxygen, carbon-sulfur, carbon-nitrogen bonds, which may also be present in the molecule. Labile also means cleavable.

Labile Linkage

A labile linkage is a chemical compound that contains a labile bond and provides a link or spacer between two other groups. The groups that are linked may be chosen from compounds such as biologically active compounds, membrane active compounds, compounds that inhibit membrane activity, functional reactive groups, monomers, and cell targeting signals. The spacer group may contain chemical moieties chosen from a group that includes alkanes, alkenes, esters, ethers, glycerol, amide, saccharides, polysaccharides, and heteroatoms such as oxygen, sulfur, or nitrogen. The spacer may be electronically neutral, may bear a positive or negative charge, or may bear both positive and negative charges with an overall charge of neutral, positive or negative.

pH-Labile Linkages and Bonds

pH-labile refers to the selective breakage of a covalent bond under acidic conditions (pH<7). That is, the pH-labile bond may be broken under acidic conditions in the presence of other covalent bonds without their breakage.

Cleavable Polymers

For inhibitor complexes, the inhibitor must be dissociated from components of the complex in the cell in order for the inhibitor to be active. This dissociation may occur outside the cell, within cytoplasmic vesicles or organelles (i.e. endosomes), in the cytoplasm, or in the nucleus. We have developed bulk polymers prepared from disulfide bond containing co-monomers and cationic co-monomers to better facilitate this process. These polymers have been shown to condense polynucleotides, and to release the nucleotides after reduction of the disulfide bond. These polymers can be used to effectively complex with nucleic acids and can also protect the nucleic acid from nucleases during delivery to the liver and other organs. After delivery to the cells the polymers are reduced to monomers, effectively releasing the nucleic acid. For instance, the disulfide bonds may be reduced by glutathione which is present in higher concentrations inside the cell. Negatively charged polymers can be fashioned in a similar manner, allowing the condensed nucleic acid particle to be “recharged” with a cleavable anionic polymer resulting in a particle with a net negative charge that after reduction of disulfide bonds will release the nucleic acid. The reduction potential of the disulfide bond in the reducible co-monomer can be adjusted by chemically altering the disulfide bonds environment. Therefore one can construct particles whose release characteristics can be tailored so that the nucleic acid is released at the proper point in the delivery process.

pH Cleavable Polymers for Intracellular Compartment Release

A cellular transport step that has importance for endocytosed inhibitor delivery is that of release from intracellular compartments such as endosomes (early and late), lysosomes, phagosomes, vesicle, endoplasmic reticulum, golgi apparatus, trans golgi network (TGN), and sarcoplasmic reticulum. Release includes movement out of an intracellular compartment into cytoplasm or into an organelle such as the nucleus. Compounds which may aid in this release include chemicals such as chloroquine, bafilomycin or Brefeldin A1. Chloroquine decreases the acidification of the endosomal and lysosomal compartments but also affects other cellular functions. Brefeldin A, an isoprenoid fungal metabolite, collapses reversibly the Golgi apparatus into the endoplasmic reticulum and the early endosomal compartment into the trans-Golgi network (TGN) to form tubules. Bafilomycin A₁, a macrolide antibiotic, is a more specific inhibitor of endosomal acidification and vacuolar type H⁺-ATPase. The ER-retaining signal (KDEL sequence) has been proposed to enhance delivery to the endoplasmic reticulum and prevent delivery to lysosomes.

To increase the stability of inhibitor particles in serum, we have recharged positively charged inhibitor/polycation complexes with polyanions that form a third layer in the inhibitor complex and make the particle negatively charged. To assist in the disruption of the inhibitor complexes, we have synthesized polymers that are cleaved in the acid conditions found in the endosome, pH 5-7. Cleavage within intracellular vesicles of polymers in the complexes may assist in vesicle disruption and release of inhibitor into the cytoplasm.

A polyion may be cleaved either by cleavage of the polymer backbone, resulting in smaller polyions, or cleavage of the link between the polymer backbone and ion containing side chain groups, resulting in small ionized molecules and a polymer. In either case, the interaction between the polyion and nucleic acid is broken and the number of molecules in the vesicle increases. This increase causes an osmotic shock which disrupts the vesicle. If the polymer backbone is hydrophobic it may interact with the membrane of the vesicle.

To construct cleavable polymers, one may attach the ions or polyions together with bonds that are inherently labile such as disulfide bonds, diols, diazo bonds, ester bonds, sulfone bonds, acetals, ketals, enol ethers, enol esters, imines, imminiums, and enamines. Another approach is construct the polymer in such a way as to put reactive groups, i.e. electrophiles and nucleophiles, in close proximity so that reaction between the function groups is rapid. Examples include having carboxylic acid derivatives (acids, esters, amides) and alcohols, thiols, carboxylic acids or amines in the same molecule reacting together to make esters, thiol esters, acid anhydrides or amides.

The present invention additionally provides for the use of polymers containing silicon-nitrogen (silazanes) linkages (either in the main chain of the polymer or in a side chain of the polymer) that are susceptible to hydrolysis. Hydrolysis of a silazane leads to the formation of a silanol and an amine. Silazanes are inherently more susceptible to hydrolysis than are silicon-oxygen-carbon linkages. The rate of hydrolysis is increased under acidic conditions. The substitution on both the silicon atom and the amine can affect the rate of hydrolysis due to steric and electronic effects. This allows for the possibility of tuning the rate of hydrolysis of the silizane by changing the substitution on either the silicon or the amine to facilitate the desired affect.

In one embodiment, ester acids and amide acids that are labile in acidic environments (pH less than 7, greater than 4) to form an alcohol and amine and an anhydride are use in a variety of molecules and polymers that include peptides, lipids, and multimolecular associations such as liposomes.

In one embodiment, ketals that are labile in acidic environments (pH less than 7, greater than 4) to form a diol and a ketone are use in a variety of molecules and polymers that include peptides, lipids, and liposomes.

In one embodiment, acetals that are labile in acidic environments (pH less than 7, greater than 4) to form a diol and an aldehyde are use in a variety of molecules and polymers that include peptides, lipids, and liposomes.

In one embodiment, enols that are labile in acidic environments (pH less than 7, greater than 4) to form a ketone and an alcohol are use in a variety of molecules and polymers that include peptides, lipids, and liposomes.

In one embodiment, iminiums that are labile in acidic environments (pH less than 7, greater than 4) to form an amine and an aldehyde or a ketone are use in a variety of molecules and polymers that include peptides, lipids, and liposomes.

pH-Sensitive Cleavage of Peptides and Polypeptides

In one embodiment, peptides and polypeptides (both referred to as peptides) are modified by an anhydride. The amine (lysine), alcohol (serine, threonine, tyrosine), and thiol (cysteine) groups of the peptides are modified by the an anhydride to produce an amide, ester or thioester acid. In the acidic environment of the internal vesicles (pH less than 6.5, greater than 4.5) (early endosomes, late endosomes, or lysosome) the amide, ester, or thioester is cleaved displaying the original amine, alcohol, or thiol group and the anhydride.

A variety of endosomolytic and amphipathic peptides can be used in this embodiment. A positively-charged amphipathic/endosomolytic peptide is converted to a negatively-charged peptide by reaction with the anhydrides to form the amide acids and this compound is then complexed with a polycation condensed nucleic acid. After entry into the endosomes, the amide acid is cleaved and the peptide becomes positively charged and is no longer complexed with the polycation-condensed nucleic acid and becomes amphipathic and endosomolytic. In one embodiment the peptides contains tyrosines and lysines. In yet another embodiment, the hydrophobic part of the peptide (after cleavage of the ester acid) is at one end of the peptide and the hydrophilic part (e.g. negatively charged after cleavage) is at another end. The hydrophobic part could be modified with a dimethylmaleic anhydride and the hydrophilic part could be modified with a citranconyl anhydride. Since the dimethylmaleyl group is cleaved more rapidly than the citrconyl group, the hydrophobic part forms first. In another embodiment the hydrophilic part forms alpha helixes or coil-coil structures.

pH-Sensitive Cleavage of Lipids and Liposomes

In another embodiment, the ester, amide or thioester acid is complexed with lipids and liposomes so that in acidic environments the lipids are modified and the liposome becomes disrupted, fusogenic or endosomolytic. The lipid diacylglycerol is reacted with an anhydride to form an ester acid. After acidification in an intracellular vesicle the diacylglycerol reforms and is very lipid bilayer disruptive and fusogenic.

Reporter Molecules

There are three types of reporter (marker) gene products that are expressed from reporter genes. The reporter gene/protein systems include:

a) Intracellular gene products such as luciferase, β-galactosidase, or chloramphenicol acetyl transferase. Typically, they are enzymes whose enzymatic activity can be easily measured.

b) Intracellular gene products such as β-galactosidase or green fluorescent protein which identify cells expressing the reporter gene. On the basis of the intensity of cellular staining, these reporter gene products also yield qualitative information concerning the amount of foreign protein produced per cell.

c) Secreted gene products such as secreted alkaline phosphatase (SEAP), growth hormone, factor IX, or alpha1-antitrypsin are useful for determining the amount of a secreted protein that a gene transfer procedure can produce. The reporter gene product can be assayed in a small amount of blood.

### EXAMPLES

### 1. Inhibition of Luciferase Gene Expression by siRNA in Liver Cells In Vivo

Single-stranded, gene-specific sense and antisense RNA oligomers with overhanging 3′ deoxyribonucleotides were prepared and purified by PAGE. The two oligomers, 40 μM each, were annealed in 250 μl buffer containing 50 mM Tris-HCl, pH 8.0 and 100 mM NaCl, by heating to 94° C. for 2 minutes, cooling to 90° C. for 1 minute, then cooling to 20° C. at a rate of −20° C. per minute. The resulting siRNA was stored at −20° C. prior to use.

The sense oligomer with identity to the luc+ gene has the sequence: SEQ ID NO: 4 5′-rCrUrUrArCrGrCrUrGrArGrUrArCrUrUrCrGrATT-3′, which corresponds to positions 155-173 of the luc+ reading frame. The letter “r” preceding a nucleotide indicates that nucleotide is a ribonucleotide. The antisense oligomer with identity to the luc+ gene has the sequence: SEQ ID NO: 5 5′-rUrCrGrArArGrUrArCrUrCrArGrCrGrUrArArGTT-3′, which corresponds to positions 155-173 of the luc+ reading frame in the antisense direction. The letter “r” preceding a nucleotide indicates that nucleotide is a ribonucleotide. The annealed oligomers containing luc+ coding sequence are referred to as siRNA-luc+.

The sense oligomer with identity to the ColE1 replication origin of bacterial plasmids has the sequence: SEQ ID NO: 6 5′-rGrCrGrArUrArArGrUrCrGrUrGrUrCrUrUrArCTT-3′. The letter “r” preceding a nucleotide indicates that nucleotide is a ribonucleotide. The antisense oligomer with identity to the ColE1 origin of bacterial plasmids has the sequence: SEQ ID NO: 7 5′-rGrUrArArGrArCrArCrGrArCrUrUrArUrCrGrCTT-3′. The letter “r” preceding a nucleotide indicates that nucleotide is a ribonucleotide. The annealed oligomers containing ColE1 sequence are referred to as siRNA-ori.

Plasmid pMIR48 (10 μg), containing the luc+ coding region (Promega Corp.) and a chimeric intron downstream of the cytomegalovirus major immediate-early enhancer/promoter, was mixed with 0.5 or 5 μg siRNA-luc+, diluted in 1-3 ml Ringer's solution (147 mM NaCl, 4 mM KCl, 1.13 mM CaCl₂) and injected into the tail vein of ICR mice over 7-120 seconds. One day after injection, the livers were harvested and homogenized in lysis buffer (0.1% Triton X-100, 0.1 M K-phosphate, 1 mM DTT, pH 7.8). Insoluble material was cleared by centrifugation. 10 μl of the cellular extract or extract diluted 10× was analyzed for luciferase activity using the Enhanced Luciferase Assay kit (Mirus).

Co-injection of 10 μg pMIR48 and 0.5 μg siRNA-luc+ results in 69% inhibition of Luc+ activity as compared to injection of 10 μg pMIR48 alone. Co-injection of 5 μg siRNA-luc+ with 10 μg pMIR48 results in 93% inhibition of Luc+ activity.

### 2. Inhibition of Luciferase Expression by siRNA is Gene Specific in Liver In Vivo

Two plasmids were injected simultaneously either with or without siRNA-luc+ as described in Example 1. The first plasmid, pGL3 control (Promega Corp, Madison, Wis.), contains the luc+ coding region and a chimeric intron under transcriptional control of the simian virus 40 enhancer and early promoter region. The second, pRL-SV40, contains the coding region for the Renilla renifonnis luciferase under transcriptional control of the Simian virus 40 enhancer and early promoter region.

10 μg pGL3 control and 1 μg pRL-SV40 was injected as described in Example 1 with 0, 0.5 or 5.0 μg siRNA-luc+. One day after injection, the livers were harvested and homogenized as described in Example 1. Luc+ and Renilla Luc activities were assayed using the Dual Luciferase Reporter Assay System (Promega). Ratios of Luc+ to Renilla Luc were normalized to the no siRNA-Luc+ control. siRNA-luc+ specifically inhibited the target Luc+ expression 73% at 0.5 μg co-injected siRNA-luc+ and 82% at 5.0 μg co-injected siRNA-luc+.

### 3. Inhibition of Luciferase Expression by siRNA is Gene Specific and siRNA Specific in Liver In Vivo

10 μg pGL3 control and 1 μg pRL-SV40 were injected as described in Example 1 with either 5.0 μg siRNA-luc+ or 5.0 control siRNA-ori. One day after injection, the livers were harvested and homogenized as described in Example 1. Luc+ and Renilla Luc activities were assayed using the Dual Luciferase Reporter Assay System (Promega). Ratios of Luc+ to Renilla Luc were normalized to the siRNA-ori control. siRNA-Luc+ inhibited Luc+ expression in liver by 93% compared to siRNA-ori indicating inhibition by siRNAs is sequence specific in this organ.

### 4. In Vivo Delivery of siRNA by Increased-Pressure Intravascular Injection Results in Strong Inhibition of Target Gene Expression in a Variety of Organs

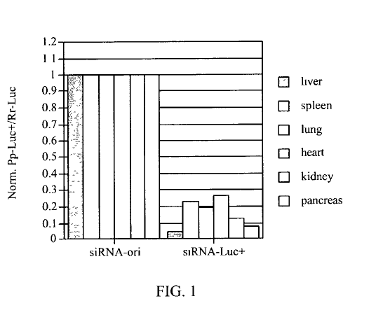

10 μg Rg pGL3 Control and 1 μg pRL-SV40 were co-injected with 5 μg siRNA-Luc+ or 5 μg control siRNA (siRNA-ori) targeted to sequence in the plasmid backbone as in example 1. One day after injection, organs were harvested and homogenized and the extracts assayed for target firefly luciferase+ activity and control Renilla luciferase activity. Firefly luciferase+ activity was normalized to that Renilla luciferase activity in order to compensate for differences in transfection efficiency between animals. Results are shown in FIG. 1. Expression of firefly luciferase+ activity was strongly inhibited in liver (95% inhibition), spleen (77%), lung (81%), heart (74%), kidney (87%) and pancreas (92%), compared to animals injected with the control siRNA-on. Animals injected with plasmid alone contained similar luciferase activities to those injected with the control siRNA-ori alone, indicating that the presence of siRNA alone does not significantly affect in vivo plasmid DNA transfection efficiencies (data not shown).

These results (FIG. 1) indicate effective delivery of siRNA to a number of different tissue types in vivo. Furthermore, the fact that expression of the control Renilla luciferase was not affected by the presence of siRNA suggests that siRNA is not inducing an interferon response. This is the first demonstration of the effectiveness of siRNA for inhibiting gene expression in post-embryonic mammalian tissues and demonstrates siRNA could be delivered to these organs to inhibit gene expression.

### 5. Inhibition of Luciferase Expression by siRNA is Gene Specific and siRNA Specific in Liver after Bile Duct Delivery In Vivo

10 μg pGL3 control and 1 μg pRL-SV40 with 5.0 μg siRNA-luc+ or 5.0 siRNA-ori were injected into the bile duct of mice. A total volume of 1 ml in Ringer's buffer was delivered at 6 ml/min. The inferior vena cava was clamped above and below the liver before injection and clamps were left on for two minutes after injection. One day after injection, the liver was harvested and homogenized as described in Example 1. Luc+ and Renilla Luc activities were assayed using the Dual Luciferase Reporter Assay System (Promega). Ratios of Luc+ to Renilla Luc were normalized to the siRNA-ori control. siRNA-Luc+ inhibited Luc+ expression in liver by 88% compared to the control siRNA-ori.

### 6. Inhibition of Luciferase Expression by siRNA is Gene Specific and siRNA Specific in Muscle In Vivo after Arterial Delivery

10 μg pGL3 control and 1 μg pRL-SV40 with 5.0 μg siRNA-luc+ or 5.0 siRNA-ori were injected into iliac artery of rats under increased pressure. Specifically, animals were anesthetized and the surgical field shaved and prepped with an antiseptic. The animals were placed on a heating pad to prevent loss of body heat during the surgical procedure. A midline abdominal incision will be made after which skin flaps were folded away and held with clamps to expose the target area. A moist gauze was applied to prevent excessive drying of internal organs. Intestines were moved to visualize the iliac veins and arteries. Microvessel clips were placed on the external iliac, caudal epigastric, internal iliac, deferent duct, and gluteal arteries and veins to block both outflow and inflow of the blood to the leg. An efflux enhancer solution (e.g., 0.5 mg papaverine in 3 ml saline) was injected into the external iliac artery though a 25 g needle, followed by the plasmid DNA and siRNA containing solution (in 10 ml saline) 1-10 minutes later. The solution was injected in approximately 10 seconds. The microvessel clips were removed 2 minutes after the injection and bleeding was controlled with pressure and gel foam. The abdominal muscles and skin were closed with 4-0 dexon suture.

Four days after injection, rats were sacrificed and the quadriceps and gastrocnemius muscles were harvested and homogenized as described in Example 1. Luc+ and Renilla Luc activities were assayed using the Dual Luciferase Reporter Assay System (Promega). Ratios of Luc+ to Renilla Luc were normalized to the siRNA-ori control. siRNA-Luc+ inhibited Luc+ expression in quadriceps and gastrocnemius by 85% and 92%, respectively, compared to the control siRNA-ori.

### 7. RNAi of SEAP Reporter Gene Expression using siRNA In Vivo

Single-stranded, SEAP-specific sense and antisense RNA oligomers with overhanging 3′ deoxyribonucleotides were prepared and purified by PAGE. The two oligomers, 40 μM each, were annealed in 250 μl buffer containing 50 mM Tris-HCl, pH 8.0 and 100 mM NaCl, by heating to 94° C. for 2 min, cooling to 90° C. for 1 min, then cooling to 20° C. at a rate of 1° C. per min. The resulting siRNA was stored at −20° C. prior to use.

The sense oligomer with identity to the SEAP reporter gene has the sequence: SEQ ID NO: 8 5′-rArGrirGrCrArArCrUrUrCrCrArGrArCrCrArUTT-3′, which corresponds to positions 362-380 of the SEAP reading frame in the sense direction. The letter “r” preceding a nucleotide indicates that nucleotide is a ribonucleotide. The antisense oligomer with identity to the SEAP reporter gene has the sequence: SEQ ID NO: 9 5′-rArUrGrGrUrCrUrGrGrArArGrUrUrGrCrCrCrUTT-3′, which corresponds to positions 362-380 of the SEAP reading frame in the antisense direction. The letter “r” preceding a nucleotide indicates that nucleotide is a ribonucleotide. The annealed oligomers containing SEAP coding sequence are referred to as siRNA-SEAP.

Plasmid pMIR141 (10 μg), containing the SEAP coding region under transcriptional control of the human ubiquitin C promoter and the human hepatic control region of the apolipoprotein E gene cluster, was mixed with 0.5 or 5 μg siRNA-SEAP or 5 μg siRNA-ori, diluted in 1-3 ml Ringer's solution (147 mM NaCl, 4 mM KCl, 1.13 mM CaCl), and injected into the tail vein over 7-120 seconds. Control mice also included those injected with pMIR141 alone. Each mouse was bled from the retro-orbital sinus one day after injection. Cells and clotting factors were pelleted from the blood to obtain serum. The serum was then evaluated for the presence of SEAP by a chemiluminescence assay using the Tropix Phospha-Light kit. Results showed that SEAP expression was inhibited by 59% when 0.5 μg siRNA-SEAP was delivered and 83% when 5.0 μg siRNA-SEAP was delivered. No decrease in SEAP expression was observed when 5.0 μg siRNA-ori was delivered indicating the decrease in SEAP expression by siRNA-SEAP was gene specific.

TABLE 1
Inhibition of SEAP expression in vivo following delivery by tail vain
injection of SEAP expression plasmid and siRNA-SEAP.

| injection | Ave. SEAP (ng/ml) | St. Dev. |
|---|---|---|
| plasmid only | 2239 | 1400 |
| siRNA-ori (5.0 μg) | 2897 | 1384 |
| siRNA-SEAP (0.5 μg) | 918 | 650 |
| siRNA-SEAP (5.0 μg) | 384 | 160 |

### 8. Inhibition of Green Fluorescent Protein in Transgenic Mice using siRNA

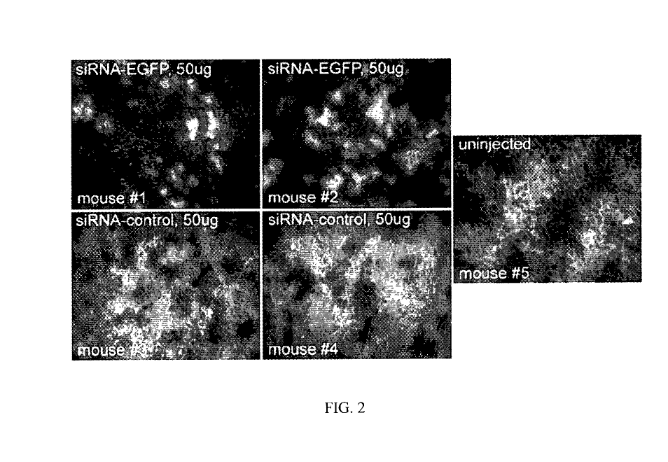

The commercially available mouse strain C57BL/6-TgN(ACTbEGFP)10sb (The Jackson Laboratory) has been reported to express enhanced green fluorescent protein (EGFP) in all cell types except erythrocytes and hair²⁴. These mice were injected with siRNA targeted against EGFP (siRNA-EGFP) or a control siRNA (siRNA-control) using the increased pressure tail vein intravascular injection method described previously. 30 h post-injection, the animals were sacrificed and sections of the liver were prepared for fluorescence microscopy. Liver sections from animals injected with 50 μg siRNA-EGFP displayed a substantial decrease in the number of cells expressing EGFP compared to animals injected with siRNA-control or mock injected (FIG. 2). The data shown here demonstrate effective delivery of siRNA-EGFP to the liver. The delivered siRNA-EGFP then inhibited EGFP gene expression in the mice. We have therefore shown the ability of siRNA to inhibit the expression of an endogenous gene product in post-natal mammals.

### 9. Inhibition of Endogenous Mouse Cytosolic Alanine Aminotransferase (ALT) Expression After In Vivo Delivery of siRNA

Single-stranded, cytosolic alanine aminotransferase- specific sense and antisense RNA oligomers with overhanging 3′ deoxyribonucleotides were prepared and purified by PAGE. The two oligomers, 40 μM each, were annealed in 250 μl buffer containing 50 mM Tris-HCl, pH 8.0 and 100 mM NaCl, by heating to 94° C. for 2 minutes, cooling to 90° C. for 1 minute, then cooling to 20° C. at a rate of 1° C. per minute. The resulting siRNA was stored at −20° C. prior to use. The sense oligomer with identity to the endogenous mouse and rat gene encoding cytosolic alanine aminotransferase has the sequence: SEQ ID NO: 10 5′-rCrArCrUrCrArGrUrCrUrCrUrArArGrGrCrUTT-3′, which corresponds to positions 928-946 of the cytosolic alanine aminotransferase reading frame in the sense direction. The letter “r” preceding a nucleotide indicates that nucleotide is a ribonucleotide. The sense oligomer with identity to the endogenous mouse and rat gene encoding cytosolic alanine aminotransferase has the sequence: SEQ ID NO: 11 5′-rArGCrCrCrUrUrArGrArGrArCrUrGrArGrUrGTT-3′, which corresponds to positions 928-946 of the cytosolic alanine aminotransferase reading frame in the antisense direction. The letter “r” preceding a nucleotide indicates that nucleotide is a ribonucleotide. The annealed oligomers containing cytosolic alanine aminotransferase coding sequence are referred to as siRNA-ALT

Mice were injected into the tail vein over 7-120 seconds with 40 μg siRNA-ALT diluted in 1-3 ml Ringers solution (147mM NaCl, 4mM KCl 1.13mM CaCl₂). Control mice were injected with Ringer's solution without siRNA. Two days after injection, the livers were harvested and homogenized in 0.25 M sucrose. ALT activity was assayed using the Sigma diagnostics INFINITY ALT reagent according to the manufacturers instructions. Total protein was determined using the BioRad Protein Assay. Mice injected with 40 μg siRNA-ALT had an average decrease in ALT specific activity of 32% compared to mice injected with Ringer's solution alone.

### 10. Inhibition of Expression of Virally Expressed Luciferase in Mammalian Cells in Culture by siRNA

HeLa cells in culture were first infected with adenovirus containing the luciferase gene under control of the phosphoglycerol kinase (PGK) enhancer/promoter (Ad2PGKluciferase). Infection of HeLa cells with Ad2PGKluciferase resulted in expression of luciferase in this cell line. After infection, siRNA targeted to the luciferase coding region or control siRNAs were delivered to the cells and the amount of luciferase activity was determined 24 h later.

HeLa cells were seeded to 50% confluency in Dulbecco's Modified Eagle's Medium (DMEM) supplemented with 10% fetal bovine serum (FBS) in a T25 flask and incubated in a 5% CO₂ humidified incubator at 37° C. 16 h later, cells were washed with PBS, trypsinized, harvested and resuspended in 13 ml DMEM/10% FBS. 500 μl of the cell suspension was distributed to each well in a 24 well plate. After 16 h incubation, the media in each well was replaced with 100 μl DMEM/10% FBS containing 5 μl Ad2PGKluciferase (2.5×10¹⁰ particles/ml stock). After incubation for 2 h, 400 μl DMEM/10% FBS was added to each well followed by the addition of siRNA complexed with TransIT-TKO (Mirus Corporation). For preparation of the siRNA complexes 7.5 μg Trans IT-TKO was diluted in 50 μl serum-free Opti-MEM and incubated at room temperature for 5 minutes. siRNA was added in order to give a final concentration of siRNA per well of 0, 1, 10 or 100 nM and incubated for 5 minutes at room temperature. Complexes were then added directly to the wells. SiRNAs targeted to the either luciferase gene, the luciferase⁺ gene, or an unrelated gene product were used (siRNA-Luc, siRNA-Luc⁺, and siRNA-c respectively). Only siRNA-Luc contained sequence identical to Ad2PGKluciferase. All assay points were performed in duplicate wells.

24 hours after delivery of siRNA, cells were lysed and luciferase activity was assayed. Results indicate that luciferase activity was inhibited 35% at 1 nM siRNA-Luc and 53% at 10 nM siRNA-Luc (Table 2). No inhibition was observed using either siRNA-Luc⁺, which contains three base pair mismatches relative to siRNA-luc or siRNA-c. These results demonstrate that siRNA can be used to inhibit expression of a virally encoded gene. In addition, the fact that siRNA-luc⁺ was unable to inhibit luciferase expression demonstrates that siRNA-mediated RNAi exhibits high sequence specificity. This example provides proof-of-principle that siRNA can be used to inhibit the expression of viral gene products in a sequence-specific manner.

TABLE 2
SiRNA-mediated RNA interference of virally encoded luciferase in HeLa
cells.

| | % Luciferase activity | | |
|---|---|---|---|
| [siRNA] | siRNA-Luc | siRNA-Luc⁺ | siRNA-c |
| 0 nM | 100 | NA | NA |
| 1 nM | 65 | 101 | 91 |
| 10 nM | 47 | 117 | 129 |

### 11. Delivery of siRNA and Morpholino Antisense Oligonucleotide to Mammalian HeLa Cells Simultaneously

HeLa cells were maintained in Dulbecco's Modified Eagle Medium supplemented with 10% fetal bovine serum. All cultures were maintained in a humidified atmosphere containing 5% CO₂ at 37° C. Approximately 24 hours prior to transfection, cells were plated at an appropriate density in a T75 flask and incubated overnight. At 50% confluency, cells were initially transfected with pGL3 control (firefly luciferase, Promega, Madison Wis.) and pRL-SV40 (sea pansy luciferase, Promega, Madison, Wis.) using TransIT-LT1 transfection reagent according to the manufacturer's recommendations (Mirus Corporation, Madison, Wis.). 15 μg pGL3 control and 50 ng pRL-SV40 were added to 45 μl TransIT-LT1 in 500 μl Opti-MEM (Invitrogen) and incubated 5 min at RT. DNA complexes were then added to cells in the T75 flask and incubated 2 h at 37° C. Cells were washed with PBS, harvested with trypsin/EDTA, suspended in media, plated into a 24-well plate with 250 μl DMEM+10% serum and incubated 2 h at 37° C. After incubation for 2 h, 400 μl DMEM/10% FBS was added to each well followed by the addition of siRNA complexed with TransIT-TKO (Mirus Corporation). For preparation of the siRNA and morpholino-containing complexes, 2 μl TransIT-TKO was diluted in 50 μl serum-free Opti-MEM and incubated at room temperature for 5 minutes. siRNA was added in order to give a final concentration of siRNA per well of 0, 0.1, or 10 nM and morpholino added to give a final concentration of morpholino per well of 0, 10, 100 or 1000 nM and incubated for 5 minutes at room temperature. Complexes were then added directly to the wells. All assay points were performed in duplicate wells.

The pGL3 control plasmid contains the firefly luc+ coding region under transcriptional control of the simian virus 40 enhancer and early promoter region. The pRL-SV40 plasmid contains the coding region for Renilla reniformis, sea pansy, luciferase under transcriptional control of the simian virus 40 enhancer and early promoter region.

Morpholino antisense molecule and siRNAs used in this example were as follows:

Morpholino-Luc (GeneTools Philomath, Oreg.), SEQ ID NO: 1 5′ TTATGTTTTTGGCGTCTFCCATGGT-3′ (Luc+ −3 to +22 of pGL3 Control Vector), was designed to base pair to the region surrounding the Luc+ start codon in order to inhibit translation of mRNA. Sequence of the start codon in the antisense orientation is underlined.

Standard control morpholino, SEQ ID NO: 10 5′-CCTCTTACCTCAGTTACAATTTATA-3′, contains no significant sequence identity to Luc+ sequence or other sequences in pGL3 Control Vector

GL3 siRNA-Luc+(nucleotides 155-173 of Luc+ coding sequence):

SEQ ID NO: 4

5′ rCrUrUrArCrGrCrUrGrArGrUrArCrUrUrCrGrAdTdT 3′

SEQ ID NO: 5

3′ dTdTrGrArArUrGrCrGrArCrUrCrArUrGrArArGrCrU 5′

Single-stranded, gene-specific sense and antisense RNA oligomers with overhanging 3′ deoxynucleotides were prepared and purified by PAGE (Dhannacon, LaFayette, Colo.). The two complementary oligonucleotides, 40 μM each, are annealed in 250 μl 100 mM NaCl/50 mM Tris-HCl, pH 8.0 buffer by heating to 94° C. for 2 minutes, cooling to 90° C. for 1 minute, then cooling to 20° C. at a rate of 1° C. per minute. The resulting siRNA was stored at −20° C. prior to use.

In order to deliver the morpholino to cells in culture using the cationic transfection reagent, TransIT-TKO (Mirus Corporation) the morpholino was first annealed to a DNA oligonucleotide of complementary sequence. The sequence of the DNA strand is as follows: SEQ ID NO: 2 5′-GCCAAAAACATAAACCATGGAAGACT-3′. The morpholino and complementary DNA oligonucleotide, 0.5 mM each, are annealed in 5 mM Hepes, pH 8.0 buffer by heating to 94° C. for 2 minutes, cooling to 90° C. for 1 minute, then cooling to 20° C. at a rate of 1° C. per minute. The resulting morpholino/DNA complex was stored at −20° C. prior to use.

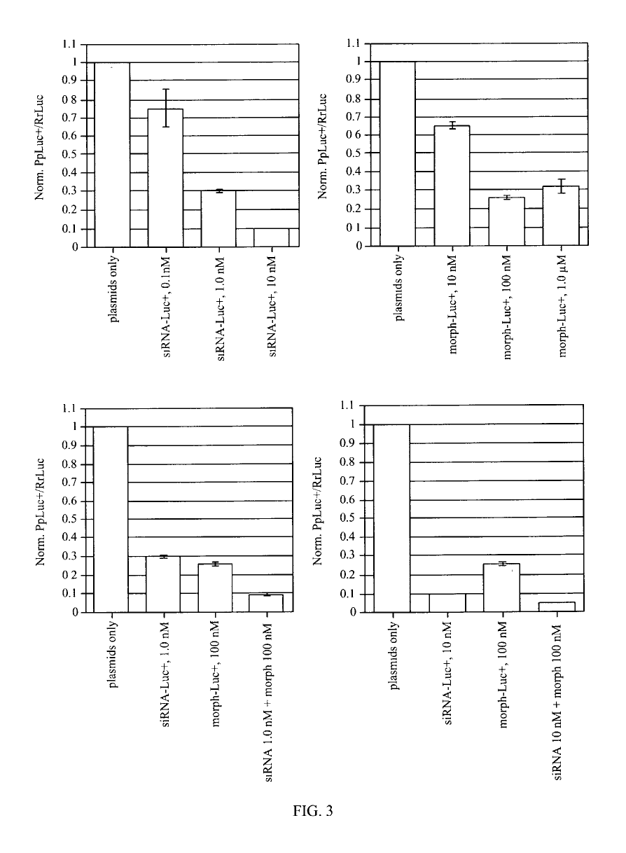

Cells were harvested after 24 h and assayed for luciferase activity using the Promega Dual Luciferase Kit (Promega). A Lumat LB 9507 (EG&G Berthold, Bad-Wildbad, Germany) luminometer was used. The amount of luciferase expression was recorded in relative light units. Numbers were then adjusted for control sea pansy luciferase expression and are expressed as the percentage of firefly luciferase expression in the absence of siRNA (FIG. 3) Numbers are the average for at least two separate wells of cells.

These data demonstrate that when siRNA and morpholino are delivery simultansously, the degree of inhibition is greater than with delivery of either siRNA or morphlino alone.

### 12. Inhibition of Luciferase Expression by Delivery of Antisense Morpholino and siRNA Simultaneously to Liver In Vivo

Morpholino antisense molecule and siRNAs used in this example were as follows:

DL94 morpholino (GeneTools Philomath, Oreg.), SEQ ID NO: 1 5′-TTATGTTTTTGGCGTCTTCCATGGT-3′ (Luc+ −3 to +22 of pGL3 Control Vector), was designed to base pair to the region surrounding the Luc+ start codon in order to inhibit translation of mRNA. Sequence of the start codon in the antisense orientation is underlined.

Standard control morpholino, SEQ ID NO: 3 5′-CCTCTTACCTCAGTTACAATTTATA -3′, contains no significant sequence identity to Luc+ sequence or other sequences in pGL3 Control Vector

GL3 siRNA-Luc+ (nucleotides 155-173 of Luc+ coding sequence):

SEQ ID NO: 4

5′ rCrUrUrArCrGrCrUrGrArGrUrArCrUrUrCrGrAdTdT 3′

SEQ ID NO: 5

3′ dTdTrGrArArUrGrCrGrArCrUrCrArUrGrArArGrCrU 5′

DL88:DL88C siRNA (targets EGFP 477-495, nt765-783):

SEQ ID NO: 12

5′ rGrArArCrGrGrCrArUrCrArArGrGrUrGrArArCdTdT 3′

SEQ ID NO: 13

3′ dTdTrCrUrUrGrCrCrCrUrArGrUrUrCrCrArCrUrUrG 5′

Two plasmid DNAs±siRNA and ±antisense morpholino in 1-3 ml Ringer's solution (147 mM NaCl, 4 mM KCl, 1.13 mM CaCl₂) were injected, in 7-120 seconds, into the tail vein of mice. The plasmids were pGL3 control, containing the luc+ coding region under transcriptional control of the simian virus 40 enhancer and early promoter region, and pRL-SV40, containing the coding region for the Renilla reniformis luciferase under transcriptional control of the Simian virus 40 enhancer and early promoter region. 2 μg pGL3 control and 0.2 μg pRL-SV40 were injected with or without 5.0 μg siRNA and with or without 50 μg DL94 morpholino. One day after injection, the livers were harvested and homogenized in lysis buffer (0.1% Triton X-100, 0.1M K-phosphate, 1 mM DTT, pH 7.8). Insoluble material were cleared by centrifugation. The homogenate was diluted 10-fold in lysis buffer and 5 μl was assayed for Luc+ and Renilla luciferase activities using the Dual Luciferase Reporter Assay System (Promega Corp.). Ratios of Luc+ to Renilla Luc were normalized to the 0 μg siRNA-Luc+ control.

TABLE 3
Inhibition of luciferase expression from pGL3 control plasmid in mouse
liver after delivery of 50 μg antisense morpholino, 5 μg siRNA or both.

| | | percent inhibition of |
|---|---|---|
| Antisense morpholino | siRNA | luciferase expression |
| — | — | 0 |
| Standard | DL88:DL88C | 0 |
| DL94 | DL88:DL88C | 85.4 ± 2.7 |
| Standard | GL3 siRNA-Luc+ | 92.0 ± 1.9 |
| DL94 | GL3 siRNA-Luc+ | 98.6 ± 0.5 |

These experiments demonstrate the near complete inhibition of gene expression in vivo when antisense morpholino is delivered together with siRNA. This level if inhibition was greater than that for either morpholino of siRNA individually.

### 13. Gene Expression using ‘recharged’ Complexes is Dependent on Polyanion Type

Transfection enhancement by adding strong polyanion to a DNA/PEI mixture was observed in both the presence and in the absence of serum. The DNA/PEI complexes were formed at final DNA concentrations of 0.02 mg/ml and 0.2 mg/ml for in vitro and in vivo studies, respectively. All complexes were formulated in buffered isotonic glucose solution (BIGS; 5% glucose, 10 mM HEPES, pH 7.5). The complexes were formed in 1.5-ml microfuge tubes by sequential addition of 5-50 μl aliquots of DNA, PEI and polyanion in their corresponding stock solutions. All polyion stock solutions were prepared in BIGS except for pDNA which was dissolved in 5 mM HEPES, 0.1 mM EDTA, pH 7.5. The tubes were vortexed for 30 s upon addition of each component. Human hepatocellular carcinoma HuH7 cells were maintained in DMEM medium supplemented with 10% fetal calf serum. The cultures were grown in a humidified atmosphere containing 5% CO₂ at 37° C. The cells were seeded in 6-well plates at 40% to 60% confluence 24 h before transfection. Before complex application, the cells were washed once with 2 ml Opti-MEM medium (Life Technologies, Inc.). The DNA complexes (2 μg/well) formulated in BIGS were added to the cells either in 2 ml Opti-MEM medium or 100% bovine serum and incubated for 4 h at 37° C. DNA-containing media were then replaced with fresh DMEM supplemented with 10% FBS. Cells were grown for an additional 48 h before they were processed for analysis of reporter gene expression.

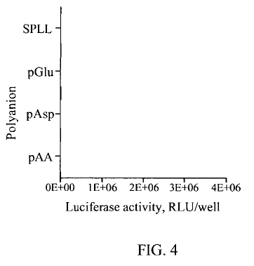

These data (FIG. 4) indicate that a polyanion, when present in the ternary complex, can enhance the release of DNA from polycation inside the cell²¹. Also, polyanions can prevent opsonization of DNA/polycation complexes with serum proteins. It is known that positively charged DNA/polycation complexes are heavily opsonized upon interaction with serum proteins (primarily serum albumin). The nature of such opsonization is believed to be primarily electrostatic²⁵. At the same time it has been found that such opsonization significantly inhibits transgene activity of DNA/brPEI complexes^{19,26}. A more significant effect was observed in the presence of serum.

### 14. Systemic Gene Transfer using Recharged DNA Complexes

Addition of high charge density polyanions to DNA/polycation complexes can enhance their gene transfer activity in vitro and in vivo. DNA/PEI complexes recharged with polyacrylic acid were tested in tail vein injections in mice using a luciferase-encoding vector. The major anatomical site of transgene expression when injecting these complexes was found to be the lung. Both linear and branched PEI (IPEI and brPEI respectively) were used as a polycation to condense plasmid DNA. However, PEI is known to be toxic when injected into animals. Therefore, the DNA/PEI complexes were recharged with PolyAcrylic Acid (pAA). In toxicity experiments, 50 μg pCILuc was condensed with 100 μg brPEI in a 5% glucose solution. DNA/PEI complexes were then recharged by addition of varying amounts of pAA, total volume of 250 μl. Toxicity of DNA/brPEI/pAA complexes was found to be dependent on the amount of pAA added to the system (−/+ratio, Table 4).

TABLE 4
Toxicity of DNA/brPEI/pAA preparations in dependence of total
polycation/polyanion ratio. DNA (pCILuc, 50 μg/animal, 3-7
animals per group) was complexed with 100 μg of brPEI and
increasing amounts of pAA in 5% glucose solution. Survival was
registered before time of sacrifice at 24 hrs post DNA injection.

| | | | | | | |
|---|---|---|---|---|---|---|
| −/+ratio | 0.49 | 0.61 | 0.67 | 0.76 | 0.84 | 0.93 |
| % survival | 0 | 39 | 42 | 76 | 71 | 100 |

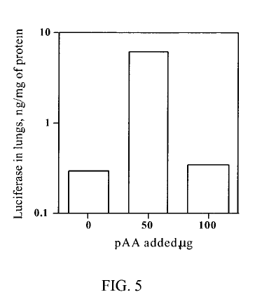

Efficacy of gene transfer to the lung was found to be optimal at 1:1 w/w DNA/pAA ratio. For determination of expression IPEI was used to condense plasmid DNA, because DNA/IPEI/TpAA complexes were more efficient in gene transfer and significantly less toxic as compared to DNA/brPEI/pAA complexes (FIG. 5). Complexes were prepares as above with 50 μg pCILuc and 200 μg IPEI and varying levels of pAA in 5% (isotonic) glucose.

All experimental animals (with recharged complexes) survived the 24 h experiment, indicating lower toxicity compared to brPEI-based complexes. Gene transfer data demonstrated characteristic bell-shaped (optimum) dependence on the amount of pAA added similar to the pattern found in vitro (data not shown). The peak gene transfer activity was also higher, generating 6 ng luciferase or 1.2×10⁸ RLU per mg of extracted protein in lungs. Available published data for similar injections of non-recharged DNA/lPEI complexes were 2 ng/mg (pCMVluc) [13] and 3×10⁷ RLU/mg (pCILuc) [11] respectively. Recharging with pAA enhanced gene transfer relative to published results without recharging. There was also more than an order of magnitude difference in transgene expression between recharged and non-recharged complexes in this experiment. This experiment was carried out at a IPEIDNA ratio of 200/50 (w/w) or nitrogen-to-phosphate (N/P) ratio of 30. Previous worked with non-recharged DNA/lPEI complexes noted increased toxicity at a N/P ratio just over 10. Hence, complex recharging with pAA allowed the use of higher N/P ratios without increasing toxicity. Recharging with a polyanion can also be done for DNA/cationic lipid complexes with similar effect on lung gene transfer and systemic toxicity (data not shown).

### 15. Second Injection of pAA Helps Further Reduce Toxicity of Recharged DNA Complexes

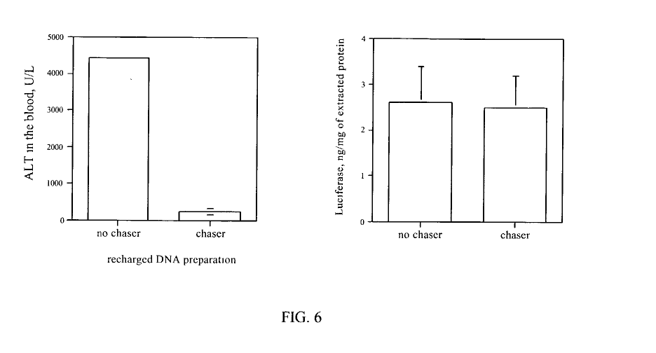

Preliminary data on lung and liver histopathology in animals systemically injected with DNA/lPEI/pAA recharged complexes revealed that such complexes still exhibit some internal toxicity (FIG. 6). In an attempt to reduce this toxicity further we performed a second “chaser” injection of polyacrylic acid (1.5 mg/animal; 30 min post DNA complex injection) while monitoring blood levels of a liver enzyme alanine aminotransferase (ALT) as an indicator of internal toxicity (see Experimental Design and Methods). Luciferase expression was also measured. The data presented on FIG. 6 clearly demonstrate that a second “chaser” polyanion injection help to significantly reduce systemic toxicity of the recharged DNA preparation while not decreasing the levels of transgene expression in lungs.

### 16. Inhibition of Luciferase Expression in Lung After In Vivo Delivery of siRNA Using Recharged Particles

Recharged particles were formed to deliver the reporter genes luciferase+ and Renilla luc as well as siRNA targeted against luciferase+ mRNA or a control siRNA to the lung. In this experiment, particles containing the reporter genes were delivered first, followed by delivery of particles containing the siRNAs. In all cases, particles were prepared with the polycation linear polyethylenimine (lPEI)and the polyanion polyacrylic acid (pAA). For delivery of reporter genes, particles were prepared which contained a mixture of the luc+ and Renilla luc expression plasmids. Normalization of expression of the two luciferase genes corrects for varying plasmid delivery efficiencies between animals. Particles containing a mixture of the expression plasmids containing the luciferase+ gene and the Renilla luciferase gene were injected intravascularly. Particles containing siRNA-Luc+ or a control siRNA were injected intravascularly immediately following injection of the plasmid-containing particles. 24 hours later, the lungs were harvested and the homogenate assayed for both Luc+ and Renilla Luc activity.

Specific experimental details were as follows: plasmid-containing particles were prepared by mixing 45 μg pGL3 control (Luc+) and 5 μg pRL-SV40 (Renilla Luc) with 300 μg lPEI in 10 mM HEPES, pH 7.5/5% glucose. After vortexing for 30 seconds, 50 μg pAA was added and the solution vortexed was for 30 seconds. siRNA-containing particles were prepared similarly, except 25 μg siRNA was used with 200 μg lPEI and 25 μg pAA. Particles containing the plasmid DNAs (total volume 250 μl) were injected into the tail vein of ICR mice. In animals that received siRNA, particles containing siRNA (total volume 100 μl) were injected into the tail vein immediately after injection of the plasmid DNA-containing particles. 1.5 mg pAA in 100 μl was then injected into the tail vein some animal 0.5 h later. 24 h later, animals were sacrificed and the lungs were harvested and homogenized. The homogenate was assayed for Luc+ and Renilla Luc activity using the Dual Luciferase Assay Kit (Promega Corporation).

Results indicate that intravascular injection of particles containing the plasmids pGL3 control and pRL-SV40 results in Luc+ and Renilla Luc expression in lung tissue (Table 2). Injection of particles containing siRNA-Luc+ after injection of the plasmid-containing particles resulted in specific inhibition of Luc+ expression. Renilla Luc expression was not inhibited. Injection of particles containing control siRNA (siRNA-c), targeted against an unrelated gene product did not result in inhibition of either Luc+ or Renilla Luc activity, demonstrating that the effect of siRNA-Luc+ on Luc+ expression is sequence specific and that injection of siRNA particles per se does not generally inhibit delivery or expression of delivered plasmid genes. These results demonstrate that particles formed with IPEI and pAA containing siRNA are able to deliver siRNA to the lung and that the siRNA cargo is biologically active once inside lung cells.

TABLE 5
Delivery of siRNA to the lung using recharged particles results in
inhibition of target gene expression.

| | | | Average | Normalized |
|---|---|---|---|---|
| | Relative light units | | Luc+/Renilla | Luc+/ |
| Particles | Replicate 1 | Replicate 2 | Luc ratio | Renilla Luc |
| plasmids only | | | | |
| Luc+ | 560994 | 680038 | 0.43 +/− | 1.00 |
| Renilla Luc | 1406188 | 1452593 | 0.05 | |
| siRNA-Luc+ | | | | |
| Luc+ | 326697 | 428079 | 0.21 +/− | 0.48 +/− |
| Renilla Luc | 1283313 | 2683842 | 0.07 | 0.16 |
| siRNA-c | | | | |
| Luc+ | 964503 | 1452962 | 0.37 +/− | 0.86 +/− |
| Renilla Luc | 2527933 | 4005381 | 0.01 | 0.03 |

### 17. In Vivo Delivery of siRNA to Mouse Liver Cells using TransIT™ In Vivo

10 μg pGL3 control and 1 μg pRL-SV40 were complexed with 11 μl TransIT™ In Vivo in 2.5 ml total volume according the manufacturer's recommendation (Mirus Corporation, Madison, Wis.). For siRNA delivery, 10 μg pGL3 control, 1 μg pRL-SV40, and either 5 μg siRNA-Luc+ or 5 μg control siRNA were complexed with 16 μl TransIT™ In Vivo in 2.5 ml total volume. Particles were injected over ˜7 s into the tail vein of 25-30 g ICR mice as described in Example 1. One day after injection, the livers were harvested and homogenized as described in Example 1. Luc+ and Renilla Luc activities were assayed using the Dual Luciferase Reporter Assay System (Promega). Ratios of Luc+ to Renilla Luc were normalized to the no siRNA control. siRNA-luc+ specifically inhibited the target Luc+ expression 96% (Table 6).

TABLE 6
Delivery of siRNA to the mouse liver using TransIT ™ In Vivo results in
inhibition of target gene expression.

| | | expression | relative LUC+ | % inhibition of |
|---|---|---|---|---|
| complex | gene | (RLUs) | expression | Luc+ expression |
| Plasmid alone | Luciferase | 31973057 | 5.1855 | 0.0 |
| | Renilla | 6165839 | | |
| Plasmid + | Luciferase | 853332 | 0.2069 | 96.0 |
| siRNA-Luc+ | Renilla | 4124726 | | |
| Plasmid + | Luciferase | 5152933 | 2.1987 | 57.5 |
| control SiRNA | Renilla | 2343673 | | |

These data show that the TransIT™ In Vivo labile polymer transfection reagent effectively delivers siRNA in vivo.

### 18. Inhibition of Vaccinia Virus in Mice

As a model for smallpox infection, the ability to attenutate vaccinia virus infection in mice by siRNA delivery was determined. Groups of 5 mice (C57B1 strain, 4-6 week old) were inoculated by installation of 20 μl of virus in PBS into each nostril with a rnicropipet, for a total volume of 40 μl containing 10⁴−10⁶ pfu of vaccinia virus (Ankara strain, GenBank accession number U94848), under isoflurane anesthesia. 5 μg E9L DNA polymerase siRNA Sequence 351: SEQ ID NO: 14 5′ rCrGrGrGrArUrArUrCrUrCrCrArGrArCrGrGrAdTdT 3′ SEQ ID NO: 15 3′ dTdTrGrCrCrCrUrArUrArGrArGrGrUrCrUrGrCrCrU 5′ was delivered at one of several time points relative to viral infection (4 hours before, simultaneous, 4 hours after, 24 hours after, 48 hours after) by injection into tail vein of mice as described above. At 1, 2, 4, and 7 days after infection, mice were sacrificed, tissue sections were collected, and viral load determined in lung, liver, spleen, brain, and bone marrow. Viral pathogenicity was assessed by histology of infected tissues, measurement of viral titers in infected tissues, and mouse survival. Tissue samples embedded in OCT Tissue-Tek were frozen in liquid nitrogen and 10 μm cryosections were fixed in 2% formaldehyde. Following permeabilization with 0.1% Triton X100, sections were blocked and stained with antibodies directed against cell surface markers or viral antigens. Antibodies against CD43 were used to detect infiltrating lymphocytes, as a marker for inflammation and viral pathogenicity. Antibodies directed against vaccinia virus proteins (e.g., A27L) were used to detect sites of viral replication. All antibodies were detected with peroxidase (Vector) or fluorescent (Sigma) secondary reagents. The amount of mRNA of the target gene and control genes were determined using the TaqMan PCR system.

The foregoing is considered as illustrative only of the principles of the invention. Furthermore, since numerous modifications and changes will readily occur to those skilled in the art, it is not desired to limit the invention to the exact construction and operation shown and described. Therefore, all suitable modifications and equivalents fall within the scope of the invention. ⁷ Player, M. R. & Torrence, P. F. The 2-5A system: modulation of viral and cellular processes through acceleration of RNA-degradation. Pharmacol Ther 78, 55-113.(1998). ⁸ Svoboda, P., Stein, P., Hayashi, H. & Schultz, R. M. Selective reduction of dormant maternal mRNAs in mouse oocytes by RNA interference. Development 127, 4147-4156. (2000). ⁹ Wianny, F. & Zernicka-Goetz, M. Specific interference with gene function by double-stranded RNA in early mouse development. Nat Cell Biol 2, 70-75. (2000). ¹⁰ Hammond, S. M., Bernstein, E., Beach, D. & Hannon, G. J. An RNA-directed nuclease mediates post-transcriptional gene silencing in Drosophila cells. Nature 404, 293-296. (2000) ¹¹ Parrish, S., Fleenor, J., Xu, S., Mello, C. & Fire, A. Functional anatomy of a dsRNA trigger. Differential requirement for the two trigger strands in RNA interference. Mol Cell 6, 1077-1087. (2000). ¹² Yang, D., Lu, H. & Erickson, J. W. Evidence that processed small dsRNAs may mediate sequence-specific mRNA degradation during RNAi in Drosophila embryos. Curr Biol 10, 1191-1200. (2000). ¹³ Zamore, P. D., Tuschl, T., Sharp, P. A. & Bartel, D. P. RNAi: double-stranded RNA directs the ATP-dependent cleavage of mRNA at 21 to 23 nucleotide intervals. Cell 101, 25-33. (2000). ¹⁴ Bernstein, E., Caudy, A. A., Hammond, S. M. & Hannon, G. J. Role for a bidentate ribonuclease in the initiation step of RNA interference. Nature 409, 363-366. (2001). ¹⁵ Minks, M. A., West, D. K., Benvin, S. & Baglioni, C. Structural requirements of double-stranded RNA for the activation of 2′,5′-oligo(A) polymerase and protein kinase of interferon-treated HeLa cells. J. Biol Chem 254, 10180-10183. (1979). ¹⁶ Manche, L., Green, S. R., Schmedt, C. & Mathews, M. B. Interactions between double-stranded RNA regulators and the protein kinase DAI. Mol Cell Biol 12, 5238-5248. (1992). ¹⁷ Caplen, N. J., Parrish S., Imani, F., Fire, A. & Morgan, R. A. Specific inhibition of gene expression by small double-stranded RNAs in invertebrate and vertebrate systems. Proc Natl Acad Sci U S A 98, 9742-9747. (2001). ¹⁸ Elbashir S M, Harborth J, Lendeckel W, Yalcin A, Weber K, Tuschl T. Duplexes of 21-nucleotide RNAs mediate RNA interference in cultured mammalian cells. Nature 411(6836), 494-498 (2001). ¹⁹ O. Boussif, F. Lezoualc'h, M. A. Zanta, M. D. Mergny, D. Scherman, B. Demencix and J. P. Behr. Proc. Natl. Acad. Sci. USA 92:7297-7301, 1995. ²⁰ V. Escriou, C. Ciolina, L. F., G. Byk, D. Scherman and P. Wils. Biochim. Biophys. Acta 1368:276-288, 1998. ²¹ Xu Y, Szoka F C Jr. Mechanism of DNA release from cationic liposome/DNA complexes used in cell transfection. Biochemistry 35(18):5616-23, 1996. ²² Bradley K A, Mogridge J, Mourez M, Collier R J, Young J A. Identification of the cellular receptor for anthrax toxin Nature. 2001 Nov 8;414(6860):225-9. ²³ Yu J Y, DeRuiter S L, Turner D L. RNA interference by expression of short-interfering RNAs and hairpin RNAs in mammalian cells. Proc Natl Acad Sci U S A 99(9):6047-6052 2002. ²⁴ Okabe M, Ikawa M, Kominami K, Nakanishi T, Nishimune Y. ‘Green mice’ as a source of ubiquitous green cells FEBS Lett May 5, 1997, 407(3):313-319. ²⁵ D. Oupicky, C. Konak, P. R. Dash, L. W. Seymour and K. Ulbrich. Bioconjug Chem 10:764-772, 1999. ²⁶ O. Boussif, M. A. Zanta and J. P. Behr. Gene Therapy 3:1074-1080, 1996.

We claim:

1. A process for delivering an inhibitor directed against an expressible nucleic acid sequence in a mammal to inhibit RNA function, comprising:
a) making the inhibitor consisting of an RNA function inhibiting sequence that is specific to the expressible nucleic acid sequence in the mammal;
b) inserting the inhibitor into a vessel in the mammal; and,
c) delivering the function inhibiting sequence to a cell wherein expression of the expressible nucleic acid sequence is inhibited.

2. The process of claim 1 wherein the inhibitor is inhibiting a viral reaction.

3. The process of claim 2 wherein the viral reaction is small pox.

4. The process of claim 1 wherein the inhibitor is inhibiting a bacterial reaction.

5. The process of claim 4 wherein the bacterial reaction is anthrax.

6. The process of claim 1 wherein the permeability of the vessel is increased.

7. The process of claim 6 wherein increasing the pressure consists of increasing a volume of fluid within the vessel.

8. The process of claim 1 wherein inhibiting RNA function comprises inhibiting translation of a messenger RNA (mRNA).

9. A complex for inhibiting nucleic acid expression in a mammal, formed by the process comprising: mixing an RNA function inhibitor and at least one compound to form a complex wherein zeta potential of the complex is less negative than the zeta potential of the inhibitor alone.

10. A complex for inhibiting nucleic acid expression in a mammal formed by the process, comprising:
a) mixing an RNA function inhibitor with a cationic first layer selected from the group consisting of polycations, proteins, amphipathic compounds, polyampholytes and nonviral vectors; wherein the complex has a positive zeta potential; and
b) recharging the complex with an anionic second layer, wherein the zeta potential is less positive than the complex with the first layer alone.
